SEQUENCE LISTING (23 sequences)

SEQ ID NO: 1 - protein, 6 aa, Unknown
  Integrin alpha subunit conserved cytoplasmic domain
IGXFFR

SEQ ID NO: 2 - protein, 7 aa, Unknown
  Integrin alpha10 subunit conserved cytoplasmic domain
ILGFFAH

SEQ ID NO: 3 - protein, 5 aa, Unknown
  Integrin alpha subunit alpha-chain motif
GFFYK

SEQ ID NO: 4 - DNA, 20 bp, Artificial Sequence
  GAPDH Forward PCR Primer
aacagcgacacccactcctc

SEQ ID NO: 5 - DNA, 23 bp, Artificial Sequence
  GAPDH Reverse PCR Primer
ggaggggagattcagtgtgtggt

SEQ ID NO: 6 - DNA, 18 bp, Artificial Sequence
  COL1A1 Forward PCR primer
gcttccctggtcttcctg

SEQ ID NO: 7 - DNA, 18 bp, Artificial Sequence
  COL1A1 Reverse PCR primer
tctcaccacggtcaccct

SEQ ID NO: 8 - DNA, 24 bp, Artificial Sequence
  COL2A1 Forward PCR Primer
gttatcgagtaccggtcacagaag

SEQ ID NO: 9 - DNA, 23 bp, Artificial Sequence
  COL2A1 Reverse PCR Primer
agtacttgggtcctttgggtttg

SEQ ID NO: 10 - DNA, 20 bp, Artificial Sequence
  Alpha1 Forward PCR Primer
tcagccaagtcaatgtttcg

SEQ ID NO: 11 - DNA, 20 bp, Artificial Sequence
  Alpha1 Reverse PCR Primer
gacccataatggcactctgc

SEQ ID NO: 12 - DNA, 20 bp, Artificial Sequence
  Alpha2 Forward PCR Primer
cgggtgtgtgttctgacatc

SEQ ID NO: 13 - DNA, 20 bp, Artificial Sequence
  Alpha2 Reverse PCR Primer
accccacctgtgtctttgtg

SEQ ID NO: 14 - DNA, 21 bp, Artificial Sequence
  Alpha10 Forward PCR Primer
tctctagaaacctccacctgg

SEQ ID NO: 15 - DNA, 24 bp, Artificial Sequence
  Alpha10 Reverse PCR Primer
ctggaaggagggctgagatgatga

SEQ ID NO: 16 - DNA, 19 bp, Artificial Sequence
  Alpha11 Forward PCR Primer
gctgcaggcagtgacagta

SEQ ID NO: 17 - DNA, 19 bp, Artificial Sequence
  Alpha11 Reverse PCR Primer
gcgatgggaatggtgatct

SEQ ID NO: 18 - DNA, 18 bp, Artificial Sequence
  Aggrecan Forward PCR Primer
cagcaccagcatcccaga

SEQ ID NO: 19 - DNA, 22 bp, Artificial Sequence
  Aggrecan Reverse PCR Primer
cagcagttgattctgattcacg

SEQ ID NO: 20 - DNA, 19 bp, Artificial Sequence
  Sox9 Forward PCR Primer
gagaacacgttccccaagg

SEQ ID NO: 21 - DNA, 20 bp, Artificial Sequence
  Sox9 Reverse PCR Primer
cgttcttcaccgacttcctc

SEQ ID NO: 22 - DNA, 20 bp, Artificial Sequence
  Versican Forward PCR Primer
agatgggttcatgggtaatt

SEQ ID NO: 23 - DNA, 24 bp, Artificial Sequence
  Versican Reverse PCR Primer
ctatacgtgcaagaaaggaacagt

(19) United States
(12) Patent Application Publication
(10) Pub. No.: US 2009/0232777 A1
(43) Pub. Date: Sep. 17, 2009

(54) Expansion and Differentiation of Mesenchymal Stem Cells

(76) Inventors: Evy Lundgren-Akerlund, Bjarred (SE); Christian Kjellman, Lovestad (SE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/281,169

(22) PCT Filed: Mar. 1, 2007
(86) PCT No.: PCT/GB2007/000731

§ 371 Date: Dec. 3, 2008

Related U.S. Application Data
(60) Provisional application No. 60/777,530, filed on Mar. 1, 2006.

(51) Int. Cl. A61K 35/12; C12N 5/02; C12Q 1/02; G01N 33/53; C12Q 1/68

(52) U.S. Cl. 424/93.7; 435/377; 435/29; 435/7.1; 435/6

(57) ABSTRACT

A cell culture system for expanding and differentiating mammalian mesenchymal stem cells to chondrocytes is provided. Said cell culture system comprises a subpopulation of isolated MSC selected for their expression of integrin alpha 10, as well as additives promoting expansion and differentiation to chondrocytes. Methods and uses of said expanded and differentiated cells with a chondrogen phenotype are also provided, as well as compositions comprising said expanded and differentiated chondrocyte cells.

### TECHNICAL FILED

This invention relates to the field of mesenchymal stem cells. More specifically it relates to a cell culture system for expanding and differentiating mammalian MSC to a chondrocyte, as well as methods and uses thereof.

### BACKGROUND OF THE INVENTION

The adult body houses so called stem cells that are capable of dividing many times while also giving rise to daughter cells with specific phenotypical characteristics. Several types of stem cells exist in the body including haematopoietic stem cells and mesenchymal stem cells. Mesenchymal stem cells (MSC) have a multilineage potential and are able to form mesenchymal tissues such as bone, cartilage, muscle, bone, ligament, fat and bone marrow stroma (Pittenger et al, Science, 284:143-147, 1999). MSC are located in bone marrow, around blood vessels, in fat, skin, muscle, bone and other tissues. Their presence contributes to the reparative capacity of these tissues.

### Medical Use of MSC

Currently, the medical use of MSC is to explore their potential in the regeneration of tissues that the body cannot naturally repair or regenerate when challenged (reviewed in Pittenger et al., Circ Res. 95:9-20, 2004). For this, MSC, are isolated, expanded in culture and stimulated to differentiate into connective tissues such as bone, cartilage, muscle, bone marrow stroma, tendon, fat and others. These tissue-engineered constructs can then be re-introduced into the human body to repair lost or damaged tissue, e.g. for cartilage or cardiac therapeutics. In another approach MSC can be directly stimulated in vivo to induce the formation of specific tissues in situ.

Having defined MSC as potential “building blocks” for tissue engineering and transplantation, researchers are now searching for better ways to make use of the MSC cells by trying to differentiate the MSC to the desired phenotype of interest—a task that has proven not to be easily solved.

Several limitations are well recognized in the art when trying to implement the use of MSC. Firstly, due to the cells, low but still appreciated, capability of triggering transplant rejections when transplanted into a patient MSC from the same patient is required, mostly. Secondly, the cells are rare and thus only a very limited amount of MSC may be retrieved from one donor. Thirdly, since the MSC has the capacity to differentiate to several types of tissue cells, e.g. cartilage, bone, muscle cells, the need for specific identification, expansion and differentiation protocols is recognised in the art to achieve a highly homogenous and specialized cell population to transplant into the patient in the need thereof.

Still a further limitation known in the art is the variation in surface molecules on human MSC seen from laboratory to laboratory. Further work is needed to see if these differences represent separate stem cell populations, reflect different cell culture techniques, or means of analysis of the cells.

WO03/106492 discloses the identification of MSC by the use of a marker specific for MSC comprising the integrin alpha 10 chain expressed on the cell surface of the MSC aiding in the identification and isolation of a highly pure population of human MSC.

WO05086845 discloses methods for maintenance of undifferentiated stem cells by exposing the cells to different proteins in growth media.

WO05113751 discloses cell culture environments for promoting MSC expansion in a serum-free cell culture system by exposing the cells to different proteins in growth media.

US2005001380 discloses a method of culturing MSC while retaining their pluripotency.

### Alpha10

A newly discovered collagen-binding integrin, alpha10beta1, includes the integrin subunit alpha10 (Camper et al., (1998) J. Biol. Chem. 273:20383-20389). The integrin is expressed on chondrocytes and shows a Mv of 160 kDa after reduction when isolated from bovine chondrocytes by collagen type I affinity purification.

Cloning and cDNA sequencing showed that it shares the general structure of other integrin alpha subunits. The predicted amino acid sequence consists of a 1167-amino acid mature protein, including a signal peptide (22 amino acids), a long extracellular domain (1099 amino acids) a transmembrane domain (22 amino acids), and a short cytoplasmic domain (22 amino acids). In contrast to most alpha-integrin subunits, the cytoplasmic domain of alpha10 does not contain the conserved sequence IGXFF(R/IC)R. Instead, the predicted amino acid sequence in alpha10 is ILGFFAH. It is suggested that the GFFYK motif in alpha-chains are important for association of integrin subunits and for transport of the integrin to the plasma membrane (De Melker et al. (1997) Biochem. J. 328:529-537).

The extracellular part contains a 7-fold repeated sequence, an I-domain (199 amino acids) and three putative divalent cation-binding site. Sequence analysis has revealed that the alpha10 subunit is most closely related to the I domain-containing [alpha] subunits with the highest identity to alpha1 (37%), alpha2 (35%) and alpha11 (42%).

### Cartilage Diseases and Repair

Cartilage may develop abnormally or may be damaged by disease, such as rheumatoid arthritis or osteoarthritis, or by trauma, each of which can lead to physical deformity and debilitation. Whether cartilage is damaged from trauma or congenital anomalies, its successful clinical regeneration is often poor.

The limited success of cartilage repair has suggested the use of MSC to repopulate and regenerate cartilage in therapeutic applications, e.g. in the treatment of cartilage conditions. The lack of cell culture systems and methods to expand and differentiate isolated MSC to a reasonable cell number with a chondrogen or chondrocyte phenotype with few or no contaminating non-chondrocytic cells has hampered the actual effect of tissue repopulation and repair of cartilage.

US20050019865 discloses cells derived from postpartum tissue, their isolation and induction of differentiation to cells of a chondrogenic or osteogenic phenotype.

It is thus highly desirable in the light of aforementioned problems to achieve a reasonable cell number of cells with a chondrocyte phenotype and/or cells with a capacity to form cartilage for repopulation and repair of cartilage. It is further highly desirable in the light of the aforementioned problems to identify and isolate a homogenous subset of mammalian MSC, such as human MSC, with a high capacity to differentiate to chondrocytes for use in repopulation and repair of cartilage. In this respect, the present invention addresses this needs and interest.

### SUMMARY OF THE INVENTION

In view of the foregoing disadvantages known in the art of tissue transplantation, repopulation and repair of cartilage the present invention provides a cell culture system for expanding and differentiating a subset of mammalian MSC to a chondrocyte for repopulation and repair of cartilage. Said chondrocyte has a high capacity to form cartilage.

One object with the present invention is thus to provide a cell culture system for differentiating mammalian MSCs to chondrocytes, the cell culture system comprising a) a population of isolated MSC, b) optionally, at least one additive promoting expansion of said mammalian MSC, and c) at least one additive promoting differentiation to chondrocytes, wherein the MSC are selected for expression of integrin alpha10.

Further embodiments are wherein the at least one additive promoting differentiation to chondrocytes is a member of the TGF beta super family proteins.

Still even further embodiments are wherein the member of the TGF beta super family is TGF beta 3.

Still further embodiments are wherein the at least one additive promoting expansion of said MSC is a FGF family protein.

Still further embodiments are wherein FGF is FGF2.

Further embodiments are wherein the MSC are selected for expression of integrin alpha10 before addition and culture of the MSC in the presence of FGF2.

Further embodiments are wherein the chondrocyte has a phenotype comprising expression of alpha10, sox9, aggrecan, and collagen II.

A second object of the present invention is to provide a method of producing a substantially homogenous population of mammalian chondrocytes, expanded and differentiated from an isolated mammalian subset of MSC, the method comprising the steps of a) providing a population of isolated MSC b) optionally culturing the isolated MSC in a) above in the presence an additive promoting expansion, and c) culturing the isolated MSC in the presence of at least one additive promoting differentiation to a chondrocyte, wherein the MSC are selected for expression of integrin alpha10 expression.

Further embodiments are wherein the MSC are selected for expression of alpha10 expression before culturing in the presence of an additive promoting expansion.

Still further embodiments are wherein the MSC are selected for expression of alpha10 expression before culturing in the presence of at least one additive promoting expansion.

Still further embodiments are wherein the MSC are selected for expression of alpha10 expression after culturing in the presence of at least one additive promoting expansion, but before culturing in the presence of an additive promoting differentiation.

A third object of the present invention is to provide an isolated and substantially homogenous cell population of expanded and differentiated mammalian MSC, wherein said MSC has a chondrocyte phenotype.

Further embodiments are wherein the chondrocyte phenotype comprises expression of alpha10, sox9, aggrecan, and collagen II.

Further embodiments are wherein the isolated and substantially homogenous population comprises at least 60, 70, 80, 90, 95, 97, 99, 99.5, or even 99.9% expanded and differentiated MSC cells with a chondrocyte phenotype.

A fourth object of the present invention is to provide a cell population according to the invention, or a cell population obtained by the method according to the invention, or a cell population obtained by the cell culture system according to the invention, for medical use.

A fifth object of the present invention is to provide uses of the cell population according to the invention, or a cell population obtained by the method according to the invention, or a cell population obtained by the cell culture system according to the invention, for the preparation of a medicament for the treatment of a cartilage condition.

Further embodiments are wherein the cartilage condition is damaged cartilage, degenerated cartilage, rheumatoid arthritis, osteoarthritis, trauma, cancer, congenital cartilage defect, or a traumatic or surgical injury.

Further objects of the present invention is to provide method for reconstituting cartilage and methods of treating a cartilage condition, as well as pharmaceutical compositions comprising an expanded and differentiated cell population according to the invention, or a cell population obtained by the method according to the invention, or a cell population obtained by the cell culture system according to the invention and a pharmaceutical acceptable carrier.

Even further objects are to provide kits, e.g. kits for expanding and differentiating isolated mammalian MSC to a chondrocyte phenotype comprising the culture system according to the invention, kits comprising expanded and differentiated cell populations according to the invention, and a kits for reconstituting cartilage.

### SHORT DESCRIPTION OF DRAWINGS

FIG. 1 shows mRNA expression of human MSCs cultures in monolayer for five days in the presence of FGF2. FGF2 cultured cells have an 8-folded increase in mRNA expression for integrin alpha 10 compared to untreated cells (A). Integrin alpha 11 showed a decreased expression after FGF2-treatment of human MSC (B).

FIG. 2A-D shows the expression of alpha10 and alpha 11 on hMSC treated with FGF2 (C and D) compared to cells not treated with FGF2 (A and B). FGF2 treatment of hMSC for 6 days resulted in an increase of alpha10 positive cells from about 12% (B) to about 70% (D). The percentage of alpha 11 positive human MSC decreased from about 95% (B) to about 58% (D). Figure A and B shows isotype control for both sets of treatment.

FIG. 3A-E shows that FGF2 pre-treated cells has an increased mRNA expression of COL2A (A), alpha10 (B), aggrecan (C), alpha 11 (D), and SOX9 (E) compared to the un-treated cells in pellet mass cultures after the cells were subjected to chondrocyte differentiation.

FIG. 4 shows that supernatans from pellet-cultures comprises newly synthesized collagen type II protein (i.e. CPII pro-peptide), thereby verifying that the FGF2 (bFGF) pre-treated hMSCs synthesize and process collagen type II (filled quadrants). The pellet cultures from un-treated hMSCs did not synthesize detectable levels of CPII pro-peptide (open triangles).

FIG. 5 shows that FGF2 (bFGF) pre-treated hMSCs have an increased proteoglycan synthesis compared to the un-treated cells and that the proteoglycan reaches a plateau around day 21.

FIG. 6 shows an overview of isolation of alpha 10 positive cells directly from bone marrow using integrin alpha 10 antibodies. Human BM cells are incubated either with integrin alpha10 antibodies (A) or an isotype control (B). Fraction C is eluted from the column as alpha 10 selected cells. Fraction D is the negative fraction, not binding to alpha 10 antibodies. Similarly, fraction E corresponds to cells that bind to isotype control and fraction F the negative fraction not binding to isotype control, merely passing through the column. All four fractions are seeded into separate 96 well plates (G and H). Fraction C is enriched for a subpopulation of MSC with an enhanced capacity to differentiate to chondrocyte cells.

FIG. 7A-M shows results from FACS analysis of MSCs isolated by plastic adherence phenotypically characterised after 21 days in monolayer culture. The results shown are from one representative bone marrow.

FIG. 8A-B shows mRNA expression of integrin α10 (A) and α11 (B) when MSCs were cultured for five days in the presence of TGFβ₃, FGF2, BMP2, BMP7 or IGF1. The results shown are from one representative experiment out of three. The mean values are calculated from triplicates and the error bars represent the SEM.

FIG. 9A-F shows the mRNA expression in MSC after culturing the cells with FGF2 for five days in monolayer cultures. MSCs were stimulated with and without FGF2 for five days in monolayer culture and analyzed for integrin α1 (B), α2 (D), α10 (A), α11 (C), β1 (E) and Sox9 (F) mRNA expression. The results shown are from one representative experiment out of three. The significance was analysed with Mann-Witneys test (N=8 parallels) and the error bars represents the SEM.

FIG. 10A-I shows protein expression of alpha10 and alpha11 and mRNA expression after MSCs were treated for 0, 1, 2, 4, and 6 days with FGF2. The expression of α10 and α11 was analysed at the protein level with FACS (A-F) and at the mRNA level using Q-PCR (G-I). The results shown are from one representative experiment.

FIG. 11A-D shows FACS results of alpha10 (A), alpha11 (B), CD105 (C) and CD166 (D) protein expression. MSCs cultured with and without FGF2 in monolayer were analysed for the expression of α10, α11 integrins as well as for CD105 (endoglin) and CD166 (ALCAM) at day 16, 28 and 50. The results shown are from one representative experiment.

FIG. 12A-C shows tiba from C57B16 (8 weeks) alpha10 KO and wt mice stained for alpha10 (B) and alpha11 (C) expression. The pictures shows the results of cryosections immunohistochemically stained for the α10 and α1 integrins and cells expressing α10 and α11 in the endosteum and periosteum. A as a control the secondary antibody was used (A). P=periosteum, BO=bone, E=endosteum and BM=bone marrow.

FIG. 13A-J shows the results of human MSCs cultured with or without FGF2 that were subjected to chondrocyte differentiation in aggregate cultures. The mRNA expression was analysed at day 7, 14 and 21 of α10 (A), α11(B), Sox9 (C), COL2A1 (D)), COL1A1 (E) aggrecan (H) and versican (I). Supernatants from day 7, 14, 21 and 28 were analyzed for newly synthesized collagen type II (CPII pro-peptide) (G). Pellets from day 7, 14, 21 and 28 were analyzed for proteoglycan synthesis using 35-S incorporation (J). The results shown are from one representative experiment and the mean value is calculated from triplicates. The error bars represent the SEM.

FIG. 14 FGF2 treated and untreated cells were tested for theirs migration potential on collagen II coated PVA-membranes in blind well chambers. The results shown are from one representative experiment out of three. The mean is calculated from 4 photos of each triplicate and the error bars represent the SD.

### DETAILED DESCRIPTION OF THE INVENTION

### Definitions

As used herein the term “mesenchymal stem cell” is intended to mean a cell that gives rise to a cell of mesenchymal lineage.

The term “expanded” is herein intended to mean that the resultant cell population is derived from an ex vivo culture of cells cultured in the presence of additives, and where the number of cultured cells exceed the number of non-cultured cells put into said culture at the starting point of the culture, i.e. before expansion.

The term “differentiated” is herein intended to mean that a resultant a cell is committed to a restricted development. A MSC differentiated to a chondrocyte is thus a cell that is committed to a chondrocytic lineage. Accordingly, “differentiation” is the process by which an unspecialized (“uncommitted”) or less specialized cell acquires the features of a specialized cell. A differentiated or differentiation induced cell is one that has taken on a more specialized (“committed”) position within the lineage of a cell.

The term “committed”, when applied to the process of differentiation, is herein intended to mean that a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. To the opposite, the term “de-differentiation refers to the process by which a cell reverts to a less specialized (or committed) position within the lineage of a cell.

As used herein, the term “lineage” of a cell defines the heredity of the cell, i.e., which cells it came from and what cells it can give rise to. The lineage of a cell places the cell within a hereditary scheme of development and differentiation.

“A lineage-specific marker” as used herein refers to a characteristic specifically associated with the phenotype of cells of a lineage of interest and can be used to assess the differentiation of an uncommitted cell to the lineage of interest.

The term “phenotype” is herein intended to mean the total characteristics of a cell or a cell population of interest, including expression of cell surface markers, both extra- and intracellular markers, as well as functional characteristics.

As used herein “functional characteristics” is herein intended to mean pertaining to the function of the cell or a cell population.

The term “desired site” is herein intended to mean a site to locate, or target, or both. The desired site further refers to a region in the host or organ that requires replacement or supplementation due to a cartilage condition. The desired site may be a single region in the organ or host, or may be multiple regions in the organ or host.

As used herein the term “cell-culture” is intended to mean the maintenance of cells in an artificial in vitro environment. It is to be understood that the term “cell culture” is a generic term and may be used to encompass the cultivation not only of individual cells but also of tissues, and organ systems.

The term “isolated” as used herein refers to a cell, cellular component, or a molecule that has been removed from its native environment.

The term about refers to an approximation of a stated value within a range of ±10%.

The term “chondrocyte” is herein intended to mean a differentiated cell responsible for secretion of extracellular matrix in cartilage. Similarly, a cell with a chondrogen phenotype or chondrocytic phenotype shows most or all characteristics of a chondrocyte.

It must be noted that as used herein and in the appended claims, the singular forms “a”, “an”, and “the” include plural reference unless the context clearly dictates otherwise. Thus, for example, reference to “a cell” includes a plurality of such cells.

### Multipotent MSC

It is known that mesenchymal stem cells (MSCs) are multipotent cells that have the capacity to differentiate into to various different lineages such as bone, cartilage and adipose tissue. MSCs are today characterized using a broad range of different cell-surface markers that are not exclusive to MSCs and not sensitive to culture conditions or differentiation capacity.

The present invention discloses that MSCs cultured in monolayer express integrin subunits α10 and α11, and that the α10 expression gradually declines during prolonged culture—indicating that α10 is a marker of immature cells. However, by culturing the MSCs in the presence of FGF2, a cytokine known to keep the mesenchymal stem cells in a more proliferative and immature state, the expression level of α10 is kept high. We also demonstrate that such MSCs can up-regulate α10 and down-regulate α11 during chondrogenic differentiation in aggregate culture. Furthermore, the present invention discloses that α10 expressing cells have a better chondrogenic differentiation potential.

In more detail, the present invention shows that the collagen binding integrins α10/β1 and α11/β1 are expressed by human MSCs monolayer cultures. It is also demonstrated that the expression of α10 increases, while α1 and α11 decrease, during aggregate culture of MSCs. α10/β1 is expressed by chondrocytes in cartilage, while α11/β1 integrins are predominantly expressed by subsets of the fibroblastic lineage. In monolayer cultures of chondrocytes, α10 expression is down-regulated and it is shown that this down-regulation is reversed by FGF2 treatment. Addition of FGF2 to chondrocytes not only results in increased α10 expression, but also in decreased all expression. FGF2 treatment of MSCs has been shown to keep the cells not only more multipotent, but also induces cell proliferation and Sox9 up-regulation. It is herein demonstrated an improved chondrogenecity as well as increased collagen-dependant migratory potential of MSCs with a high α10 expression. It is also demonstrated expression of α10 and α11 integrin subunits in the endosteum and periosteum of mice, but very low or not detectable expression levels in freshly aspired human or mouse bone marrow.

### Isolation of Mammalian MSC

The human integrin alpha 10 chain sequence is known and publicly available at GenBank™/EBI Data Bank accession number AF074015. Thus, new uses and methods of the integrin alpha10 chain are disclosed in the present invention.

Mammalian MSC, including human MSC, is generally isolated from bone marrow, peripheral blood, cord blood, liver, cartilage, perichondrion, bone, periosteum or fat. The isolation is mostly based on the cells capacity to adhere to plastic culture dishes and form colonies under specific culture conditions, while the majority of bone marrow cells do not adhere nor form colonies.

Prior to culture, a large proportion of non-mesenchymal linage cells may be removed from a stem cell source by negative or positive selection. For example, large numbers of lineage-committed cells can be removed by e.g. selective magnetic bead separations or any similar method such as panning, solid phase columns, agglutination etc. In some embodiments, at least about 80%, usually at least about 70% of the non-desired and to other lineages differentiated cells may be removed prior to culture. Mononuclear cells may be collected by density gradient centrifugation and may then be cultured in tissue culture containers, plates or flasks. Furthermore, bone marrow aspirates may be seeded onto tissue culture plates or flasks. After several hours to days, non-adherent cells are washed away and the MSCs remain. Non-adherent cells may be removed after e.g. 1, 2, 3, 4 or even 5 days, once or several times on order to remove non-adherent cells. In one embodiment, non-adherent cells are removed after 4 days. Adherent, spindle shaped fibroblast-like MSCs are kept and expanded. Medium is changed regularly, such as every 3-4 day, and adherent cells are cultured till confluence.

In order to extract human bone marrow-derived mesenchymal stem cells, any conventional method used in the art for e.g. research purpose or medical treatment and the like may be used.

In order to extract stem cells from bone marrow of other mammals than human by a laboratory-method, both ends of bone (e.g. femur, tibia) may be cut, the inside of bone may be washed by a medium suitable for culturing mesenchymal stem, cells, and mesenchymal stem cells may then be obtained from the emerged medium.

MSC, e.g. human, may then further be isolated using an optional density centrifugation and found as a part of a mononuclear cell fraction layer at the density interface of 1.073 g/ml (e.g. using Percoll™, Pharmacia or Lymphoprep, Nycomed Pharma AS, Norway). Out of this mononuclear fraction, 1/10 000- 1/100 000 cells form colonies upon culture in the serum culture dishes.

Suitable protocol for isolation of mammalian MSC, without including the marker alpha10, is further given in detail in Mason J M et al (2000, Cartilage and bone regeneration using gene-enhanced tissue engineering. Clin. Orthop. 379S:S 171-178), Chu CR et al. (1997, Osteochondral repair using perichondrial cells in Clin. Orthop. 340:220-229 (2000), and Dounchis J S et al. (2000, Cartilage repair with autogenic perichondrium cell and polylactic acid grafts. Clin. Orthop. 377:248-264). Thus, known MSC isolation methods may be used, but with the introduction of a specific selection step using the MSC subset specific marker integrin alpha 10 to identify and isolate the desired subset MSC population according to the invention and to identify and select a subset of MSC with an increased capability to expand and differentiate to chondrocytes, or with an increased capability to produce cartilage.

In order to extract the MSC from periosteum, a well-known method can be used (e.g. M. Iwasaki et al., Endocrinology 132, 1603-1608 (1993); J. Fang & B. K. Hall, Developmental Biol. 180, 701-712 (1996)).

In the art several protocols are disclosed for further expansion and differentiation of cells into desired MSC lineages and tissue cells such as chondrocytes, osteocytes, adipocytes, etc., using special culture conditions with defined additives. It is not known, however, if MSC isolated and expanded in this way are a homogenous population or the role of subsets in MSC population.

It is thus disclosed herein a subset of mammalian MSC, such as human MSC, selected on integrin alpha10 expression. Such selected cells have a high potential of differentiating to a chondrocyte and/or to a cartilage producing cell under specific conditions including both expansion and differentiation in the presence of selective additives in vitro and to expand and differentiate said subset cells into a homogenous population of chondrocytes.

Examples of additives that may be used for expansion of cells according to the invention are fibroblast growth factor, FGF, proteins, such as FGF1, FGF2, e.t.c. members of the TGF beta family such as TGF beta 1 (TGFβ1), TGF beta 2 (TGFβ2), TGF beta 3 (TGFβ3), e.t.c.

In one embodiment the additive used for expansion of cells according to the invention comprises FGF2.

Examples of additives that may be used for differentiation of cells according to the invention is members of the TGF beta (β) family, such as TGF beta 1, TGF beta 2, TGF beta 3, BMPs, such as BMP-2, growth differentiation factors, GDF, such as GDF-5, or IGF-1.

In one embodiment the additive used for differentiation of cells according to the invention comprises TGF beta 3 (TGFβ3).

Table 1 gives an overview of markers relevant for human MSC with potential to differentiate to a chondrocyte in the disclosed system and methods. Thus, a chondrogen phenotype achieved after expansion and differentiation in a system or method according to the invention is phenotype wherein a chondrocyte cell is collagen 2 positive, aggrecan positive, Sox9 positive, alpha10 positive.

In a further embodiment, said chondrocyte cell is further collagen 1 negative.

In a further embodiment, said chondrocyte cell is further collagen X negative.

In a further embodiment, said chondrocyte cell is further verse can negative.

In a further embodiment, said chondrocyte is further an integrin alpha 11 low expressing cell.

TABLE 1
Markers for MSC and chondrocytes

Cell:

| | | | Suitable method |
|---|---|---|---|
| Marker: | MSC | Chondrocyte | for analysis^{a} |
| CD45 | − | − | FACS |
| CD105 | + | + | FACS |
| CD166 | + | + | FACS |
| Alpha10 | + | + | FACS, histology |
| Alpha 11 | + | + | FACS |
| Alpha1 | + | + | FACS |
| Alpha2 | + | + | FACS |
| Beta1 | + | + | FACS |
| Beta3 | + | + | FACS |
| Alpha5 | + | + | FACS |
| CD90 | + | ? | FACS |
| CD70 | + | ? | FACS |
| CD44 | + | ? | FACS |
| Collagen 2 | − | + | RT-PCR, ELISA, |
| | | | staining histology |
| Aggrecan | | + | PCR, |
| | | | proteoglycan (pg) |
| | | | synthesis, |
| | | | histology |
| | | | SaffranO staining |
| Sox9 | | + | PCR |

^{a}Example of suitable protocols are given in the description and in the experimental part of the description.

Methods to characterize expanded and differentiated cells according to the invention, include, but are not limited to, histological, morphological, biochemical and immuno-histochemical methods, different immunomethods such as e.g. ELISA, by analysing extra or intracellular cell surface markers, or by identifying factors secreted by the expanded and differentiated cell. Further, RT-PCR may be used as a convenient method for analysing protein expression at RNA levels. Several protocols for immunomethods are given in Harlow-lane (Antibodies; A laboratory manual, CSHL, 1998).

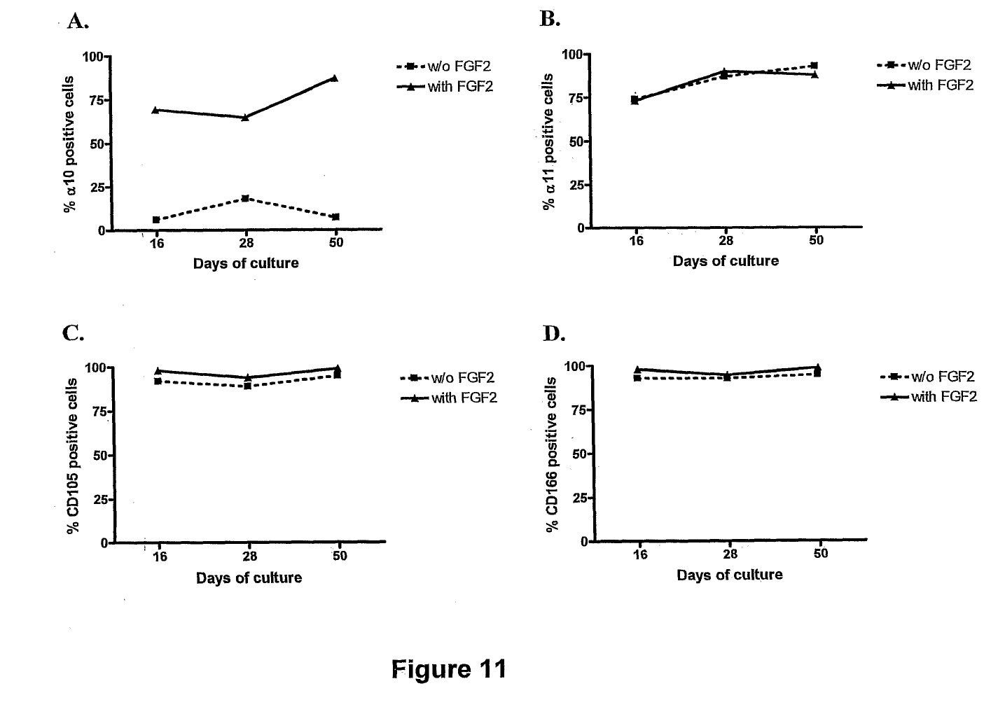

To-date, MSCs have been characterised by several different cell markers that are non-specific to MSCs and not sensitive to the state of differentiation. The co-expression of CD105 and CD166 is commonly used to define a population of mesenchymal progenitor cell. Bone marrow derived cultured MSCs as well as mesenchymal progenitor cells derived from other locations e.g articular cartilage and synovium has been shown to be CD105 and CD166 positive. However, CD105 and CD166 are not specifically expressed by progenitor cells. For example, cultured articular chondrocytes as well as skin fibroblasts has been shown to be CD105 positive. CD105 is also expressed by e.g. endothelial cells, syncytiotrophoblasts, macrophages and connective tissue stromal cells. CD166 is expressed by mesenchymal progenitor cells, but also expressed by monocytes, activated T- and B-lymphocytes and thymic epithelial cells. In addition, periosteum, the developing brain, lung and esophagus have been shown to stain positive for CD166. Monolayer cultures of human articular chondrocytes and synovial fibroblasts are also positive for CD166 (unpublished data). In the present invention approximately 10% of the bone-marrow derived mononuclear cells stain positive for CD105 and 20% of the cells express CD166. As Soon as a proliferating bone marrow derived cell population is established during culture, close to 100% of the cells are CD105 and CD166 positive. Furthermore, the presence of FGF2 does not affect the expression of these markers, even after prolonged in vitro culture (up to 50 days) (FIG. 11).

Integrin α1 (CD49a), is expressed by approximately 10% of the bone marrow derived mononuclear cells and the majority of these cells are mature hematopoietic cells. Direct selection of CD49a (α1) positive cells from bone-marrow derived MNCs has been shown to also contains cells with adipogenic, osteogenic and chondrogenic capacity. Approximately 10% of the bone marrow derived mononuclear cells stain positive for α2 (CD49b). The integrin subunits α10 or all are not detectable in bone-marrow aspirates using FACS analysis. After expansion of MSCs in culture, the cells are positive for all the collagen binding integrin subunits, i.e. al, α2, α10 and α11. The present invention shows that α10 is the only integrin alpha chain expressed by MSCs that consistently is up-regulated by FGF2. FGF2 is known to increase the proliferation rate of MSCs and it is disclosed in the present invention that it also increases their chondrogenic potential. Taken together, this makes α10 a novel cell surface marker for MSCs with high proliferative activity and sustained chondrogenic potential.

The present invention also demonstrate that integrin subunit α10 was up-regulated during chondrocytes differentiation of MSCs. Integrin subunits α1 and α11 were rapidly down-regulated during the 10 day aggregate culture of MSCs, while integrin subunit α2 expression was not affected. Partially dedifferentiated human articular chondrocytes expanded in cell cultures are positive for integrin subunits α1, α2, α10 and α11. It is known that primary chondrocytes of normal human articular cartilage express α1 and α10 but not α2. It is also known that in a mouse, a subset of chondrocytes in the deep zone are α1 positive and α1-deficient mice have an age-dependant accelerated development of OA-like lesions. Integrin subunit α10 is more uniformly expressed in chondrocytes of mouse cartilage and it is known that the α10-deficient mouse have a disturbed growth plate phenotype. Integrin α11 is not expressed in cartilage of new-born mouse limbs (data not shown). Taken together the present invention shows that the counter regulation of α10 and α11 is an excellent marker of chondrogenic differentiation.

By immunohistochemical analyses of tibia from mice, cell populations in the endosteum expressing both α10 and α11 integrin subunits was identified. It is known that the endosteum is a location where one can expect to find multipotent mesenchymal cells. However, according to the present invention, the frequency of α10 positive cells in human bone marrow is low. By FACS analysis one could not detect α10 and α11 positive cells in human bone marrow aspirates and this could be due to technical difficulties in extracting cells from the endosteum. However, considering the reported low frequency of MSCs in bone marrow (10⁻⁵-10⁻⁶), this is what we would expect if α10 is a marker of MSCs in bone marrow. Furthermore, the present invention discloses the possibility of isolating α10 positive cells directly from bone marrow and that one can enrich colony-forming units using α10 specific monoclonal antibodies.

Antibodies to alpha10 are disclosed in WO 99/51639 and in WO 2004/089990, both incorporated herein by reference.

Antibodies to alpha11 are disclosed in WO 00/75187, incorporated herein by reference.

Antibodies to both alpha11 or alpha10 may also be isolated by other methods know in the art including the use of hybridoma, recombinant, and phage display technologies, or a combination thereof. Examples are using hybridoma techniques including those known in the art and taught, for example, in Harlow et al., Antibodies: A Laboratory Manual, (Cold Spring Harbor Laboratory Press, 2nd ed. 1988) (said reference incorporated herein by reference.). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Methods for producing and screening for specific antibodies using hybridoma technology are routine and well known in the art. In a non-limiting example, mice can be immunized with a polypeptide of alpha10 or alpha11 or a cell expressing such peptide. Once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP2/0 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones. Alpha10 and alpha11 antibodies can also be generated using various phage display methods known in the art. In phage display methods, functional antibody domains are displayed on the surface of phage particles which can the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including MI3 binding domains expressed from phage with Fab, Fv or disulfide stabilized Fv antibody domains recombinantly fused to either the phage gene I11 or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57: 191-280 (1994). Libraries suitable for phage display selection are available from different companies, such as e.g. n-CoDeR® (human antibody library, BioInvent Int. AB, Sweden) or from e.g. CAT (UK).

The up-regulation of α10 by FGF2 provides a novel cell surface marker for MSCs that have a higher differentiation potential towards a chondrogenic phenotype. FGF2 has previously been shown to keep MSCs in a more undifferentiated state, with higher proliferative capacity and with longer telomeres. The present invention further discloses that that Sox9 is up-regulated during FGF2 treatment. The results of the present invention imply that α10 is a new marker expressed by multipotent mesenchymal progenitor cells. Markers defining the differentiation state or differentiation potential of MSCs are important e.g. when optimizing cultivation systems and to characterize high quality MSCs for therapy. Presently, to determine the level of differentiation, the isolated cells have to be tested in time-consuming assays for their differentiation capacity into the osteogenic, adipogenic and chondrogenic lineages. The present invention discloses that antibodies recognizing α10 as well as α11-integrins are of great value in the field of mesenchymal stem cell research.

Those skilled in the art recognize that cells with a specific cell marker, intra- or extracellular, or expression of said marker may be detected and even isolated by any means including but not limited to flow cytometric means, antibody panning, PCR, Western gel analysis, agglutination and the like. Generally, one of skill in the art must first set a detection threshold, e.g. fluorescence for flow cytometry. In setting the threshold for flow cytometry, a negative control sample population will be recorded and a gate will be set around the population of interest according to the desired forward scatter (FSC) and side scatter (SSC). The detection threshold is then adjusted so that 97% or more of the cells do not fluorescence. Once the detection threshold is set, the fluorescence of cell population of interest is recorded. A cell is considered “positive for expression” when it expresses the marker of interest at a detectable level using a specific method and defined conditions of that particular method, whether a protein or a gene. Any method may be used to determine expression such as gene expression profiles, FACS, and the like. The term “+” indicates that the cell has detectable levels of expression of the marker of interest, on protein or gene level. The term “−” indicates that the cell does not have detectable levels of expression of the marker of interest, on protein or gene level using a specific method and defined conditions of that particular method.

For flow cytometry antibodies used to detect various lineages may be conjugated to different fluorochromes. These include phycobiliproteins, e.g., phycoerythrin and allophycocyanins; fluorescein; Cy5, APC, and Texas red. Dead cells may also be detected using dyes that selectively accumulate in dead cells (e.g., propidium iodide and 7-amino 5 actinomycin D).

The expanded and differentiated alpha 10 selected subpopulation of MSC of the invention with a phenotype of a chondrocyte may also be analyzed based on gene expression profiles. In this manner, the chondrocyte or chondrogen potential may be determined. As used herein, an “expression profile” comprises one or more values corresponding to a measurement of the relative abundance of a gene expression product, including measurements of RNA levels or protein levels. Thus, the expression profile can comprise values representing the measurement of the transcriptional state or the translational state of the gene (see U.S. Pat. No. 6,040,138, U.S. Pat. No. 5,800,992, U.S. Pat. No. 6,020,135, U.S. Pat. No. 6,344,316, and U.S. Pat. No. 6,033,860).

The transcriptional state of a sample includes identifying and measuring the relative abundance of a RNA species, especially mRNAs, present in the sample. The transcriptional state can be conveniently determined by measuring transcript abundance by any of several existing technologies, such as e.g. PCR (polymerase chain reaction), microarray technology, or Northern blotting. Translational state includes identifying and measuring the relative abundance of the constituent protein species in the sample. As is known to those of skill in the art, the transcriptional state and translational state are related.

### Identification and Isolation of a Subset of MSC Using Alpha10

The identification of subsets of MSC in situ is hampered by the fact that mono specific and unique molecular probes do not exist. Since MSC has the potency of differentiating into several tissue lineages it is therefore necessary to further characterize subsets of mesenchymal stem cells to identify what subsets has the best capacity to differentiate into e.g. chondrocytes, adipocytes, osteocytes, etc. and to identify what markers or combination of markers that unequivocally identify certain desired subsets of MSC with different or unique differentiation potentials. Such markers are also useful for the isolation of said MSC subsets from e.g. bone marrow or tissue.

One subset of MSC is the subset disclosed in the present invention, with a unique expansion and differentiation potential into a chondrocyte according to methods and systems of the present invention further discussed in detail below.

WO03/106492 discloses the identification of MSC by the use of a marker specific for MSC comprising the integrin alpha 10 chain expressed on the cell surface of the MSC aiding in the identification and isolation of a highly pure population of human MSC. The published application is incorporated herein by reference.

The present invention provides the use of alpha10 as a marker for a subpopulation of MSC with enhanced chondrogenic potential.

In brief, any isolated cell population comprising MSC may be analysed for expression of the MSC specific marker integrin alpha 10 by e.g. an immuno assay known in the art such as immuno precipitation, Western blotting or flow cytometry methods, e.g. fluorescence activated cell sorting (FACS), using f.ex. a polyclonal or monoclonal antibody binding to integrin alpha 10 or any other binding entity targeting alpha 10. Examples of such are polyclonal and monoclonal antibodies made as disclosed in e.g. WO2004/9899990 (incorporated herein by reference) or according to any other in the art known technique for making monoclonal or polyclonal antibodies. One example is clone mAb 365, deposited under number DSM ACC2583 (deposited at Deutsche Sammlung von Microorganismen und Zellkulturen GmbH), or polyclonal antibodies made as disclosed in WO99516639 (incorporated herein by reference).

The identified alpha 10 expressing subpopulation of MSC may then be further selected and separated based on their integrin alpha 10 expression by any technique known in the art. Such techniques for selection are well known in the art and include various solid phase methods e.g. sorting by beads, by complement mediated lysis, by “panning” with antibody attached to a solid matrix, agglutination methods, magnetic activated cell sorting (MACS), or fluorescence activated cell sorting (FACS®).

The particular procedure for separation employed, e.g. centrifugation, mechanical separation, such as columns, membranes or bead separation, should maximize the viability of the fraction to be collected. Various techniques with different efficacy may be employed known to a person skilled in the art. The particular technique employed will depend upon efficiency of separation, cytotoxicity of the methodology, ease and speed of performance, and necessity for sophisticated equipment and/or technical skill.

In one embodiment an antibody, such as a monoclonal, polyclonal or recombinant antibody, or antibody fragment thereof, binding to alpha 10 is used. It may be attached directly or indirectly to a solid support to allow for a highly specific separation.

Procedures for separation of said subset of MSCs based on alpha 10 expression from a cell suspension aided by the methods or systems according to the invention may include magnetic separation, using e.g. antibody-coated magnetic beads, affinity chromatography based on the antibody or fragments thereof, and “panning” with an antibody or fragments thereof attached to a solid matrix, e.g., a plate, or other convenient techniques.

Other techniques providing accurate separation include fluorescence activated cell sorters by the use of e.g. an antibody or fragments thereof in the method or system according to the invention, which can have varying degrees of sophistication, e.g., a plurality of colour channels, light scattering detecting channels, impedance channels, etc. known to the skilled man in the art.

### A Cell Culture System According to the Invention

In the extra cellular matrix of cartilage, there is a pool of FGF2 bound to heparin sulphate that is released when the cartilage is damaged or subjected to loading. The present invention shows that FGF2 treated MSCs have a higher migratory capacity than untreated cells. The release of FGF2 from damaged cartilage may attract progenitor cells from the bone marrow or other stem cell pools or induce proliferation of the MSCs/chondroprogenitor cells in the damage area. It is known that human MSCs have the capacity to contract three dimensional collagen gels and that this contraction is dependant on β1 integrins. Taken together, this indicates that α10, up-regulated by FGF2, is involved in stem cell migration and homing to damaged collagen rich tissues like cartilage. Integrins are well known to be involved in cell migration, proliferation, adhesion, differentiation, control of collagen synthesis as well as MMP synthesis

According to the invention, a cell culture system for expanding and differentiating mammalian MSC to a chondrocyte is disclosed. As used herein, a chondrocyte cell is intended to include a cell with a chondrogen or chondrocytic phenotype, thus having the characteristics, including protein expression of intracellular and extracellular cell markers and functional characteristics, of a chondrocyte. Said cell culture system comprises a) a population of isolated mammalian MSC, b) optionally, at least one additive promoting expansion of said mammalian MSC, and c) at least one additive promoting differentiation to chondrocytes, wherein the population of isolated MSC is selected for expression of integrin alpha10 to select the desired subset of MSC.

In further embodiments, the at least one additive promoting differentiation to chondrocytes is a member of the TGF beta super family proteins, such as TGF beta 1, TGF beta 2 or TGF beta 3, or any other member of the TGF family, or BMP proteins such as BMP2, BMP4, BMP7, or an activin or inhibin protein.

In further embodiments, the member of the TGF beta super family is TGF beta 3.

Further embodiments include wherein the at least one additive promoting expansion of said MSC is a fibroblast growth factor, FGF, family protein, such as e.g. FGF1, or FGF2 or any other member of the FGF family.

Further embodiments are wherein FGF is FGF2.

Further embodiments include wherein additives such as e.g. FGF2 and TGFβ3, are added sequentially to said system as additives, and wherein adding of FGF2 is preceding TGFβ3 addition. Said additives will allow expansion and differentiation of the identified and selected MSC subset to a chondrocyte.

Further embodiments are wherein the MSC are selected for expression of integrin alpha10 before culturing the MSC in the presence of FGF2.

Further embodiments are wherein the MSC are selected for expression of integrin alpha10 after culturing in the presence of FGF2, but before culturing in the presence of TGFβ3.

Further embodiments are wherein the MSC are selected for expression of integrin alpha10 after culturing in the presence of TGFβ3.

Even further embodiments are wherein the chondrocyte has a phenotype comprising expression of alpha10, sox9, aggrecan, and collagen II. Said expression is protein expression and may be measured indirectly by RT-PCR at mRNA levels or directly at protein levels by e.g. any method measuring proteins, such as Western blots, ELISA, immuno precipitation, flow cytometry or similar method as described herein or known in the art.

In a further embodiment, said chondrocyte cell is further collagen 1 negative.

In a further embodiment, said chondrocyte cell is further collagen X negative.

In a further embodiment, said chondrocyte cell is further versecan negative.

In a further embodiment, said chondrocyte is further an integrin alpha 11 low expressing cell.

Further embodiments are wherein FGF2 is added in an amount of about 0.01 ng/ml to 1 μg/ml, e.g. about 0.04 ng/ml to 500 ng/ml, about 0.1 to 50 ng/ml, such as e.g. about 1, 2, 3, 4, 5, 6, 7, 8, 9, 10, 11, 12, 13 14, 15, 16, 17, 18, 19, 20, 25, 30, 35, 40, 45, or even 50 ng/ml.

In one embodiment FGF2 is added in an amount of 10 ng/ml.

Further embodiments are wherein TGFβ3 is added in an amount of about 0.01 ng/ml to 1 μg/ml, e.g. about 0.04 ng/ml to 500 ng/ml, about 0.1 to 50 ng/ml, such as e.g. about 0.1, 0.2, 0.3, 0.4, 0.5, 0.6, 0.7, 0.8, 0.9, 1, 2, 3, 4, 5, 6, 7, 8, 9, 10, 11, 12, 13 14, 15, 16, 17, 18, 19, 20, 25, 30, 35, 40, 45, or even 50 ng/ml.

In one embodiment TGFβ3 is added in an amount of 10 ng/ml.

The mammalian MSC may be any mammalian MSC such as e.g. human MSC, rat MSC, mouse MSC, horse MSC, cat MSC, dog MSC, horse MSC, camel MSC, goat MSC, cow MSC, sheep MSC, or dog MSC.

In one embodiment, said mammalian MSC are human MSC.

### A Method of Producing a Chondrocyte

According to the invention a method of producing a substantially homogenous population of mammalian chondrocytes, expanded and differentiated from an isolated mammalian subset of MSC. The method comprises the steps of a) providing a population of isolated MSC, b) optionally culturing the isolated MSC in a) above in the presence at least one additive promoting expansion, and c) culturing the isolated MSC in the presence of at least one additive promoting differentiation to a chondrocyte, wherein the isolated MSC are selected for expression of integrin alpha10 expression.

Providing the population of isolated MSC in a) above may be as described previously in the art, except form the addition that the cells are selected for the expression of integrin alpha 10. Examples are given in the paragraphs above. The population may be isolated from bone marrow (BM), peripheral blood, cord blood, liver, bone, cartilage, muscle, perichondrium, periosteum, synovial tissue, fat or any tissue comprising MSCs. The population may further be isolated from mammalian iliac crest, femora, tibiae, spine, rib or other medullary spaces. Other sources of human MSC:s include embryonic yolk sac, placenta, and umbilical cord.

In one embodiment, the population provided is isolated from BM, such as e.g. a human BM. If the population of cells is collected from BM, normally only 0.01-0.001% of the starting population, or “crude population”, are MSCs. Though, this may vary between different donors.

In one embodiment, the method for isolating a population of human MSCs further comprises the steps of
collecting bone marrow aspirate (5-30 ml) from a human patient into a syringe containing e.g. heparin to prevent clotting
washing the marrow sample with e.g. Dubecco's phosphate-buffered saline (DPBS) or any similar saline solution, and recovering the cells after centrifugation at 900 g, and repeating this procedure once more
optionally loading the cells onto 25 ml of Percoll of a density of 1.073 g/ml, or similar density gradient, in a 50-ml conical tube and separating the cells by centrifugation at 1100 g for 30 min at 20° C.,
collecting the nucleated cells from the interface, diluting with two volumes of DPBS, and collecting by centrifugation at 900 g,
resuspending the cells, counting the cells, and plating out the cells at the required density, suitable 200,000-cells/cm²,
culturing the cells in Dulbecco's modified Eagle's medium, DMEM, or any other suitable medium (low glucose) containing 10%-20% foetal bovine serum (FBS) or human serum or even without serum,
replacing the medium at 24 and 72 hours and every third or fourth day thereafter, and
sub-culturing the huMSCs that grow as symmetric colonies at 10 to 14 days by treatment with 0.05% trypsin and 0.53 mM EDTA for 5 min, rinsed from the substrate with serum-containing medium, collected by centrifugation at 800 g for 5 min, and seeded into fresh flasks at 5000 to 6000 cells/cm².

The selection of said MSC sub-population based on integrin alpha 10 expression may be done as describe in detail in other paragraphs herein.

Further embodiments are wherein the MSC wherein the MSC are selected for expression of alpha 10 expression before culturing in the presence of an additive promoting expansion.

Further embodiments are wherein the MSC are selected for expression of alpha10 expression after culturing in the presence of an additive promoting expansion, but before culturing in the presence of an additive promoting differentiation.

Further embodiments are wherein the MSC are selected for expression of alpha10 expression after culturing in the presence of an additive promoting differentiation.

Examples of additives promoting expansion and/or differentiation of mammalian MSC are given herein.

Further embodiments are wherein the additive promoting expansion is FGF2.

Further embodiments are wherein the additive promoting differentiation is TGF beta 3 (TGFβ3).

Even further embodiments are wherein the MSC are selected for expression of alpha10 expression after culturing in the presence of FGF2, but before culturing in the presence of TGFβ3.

Even further embodiments are wherein the MSC are selected for expression of alpha10 expression after culturing in the presence of TGFβ3.

Even further embodiments are wherein said culture in the presence of FGF2 is for about 1 to 40 weeks, such as e.g. 1, 2, 3, 4, 5, 6, 7, 8, 9, or even 10, 20, 30 or 40 weeks.

In one embodiment, said culture in the presence of FGF2 is for about 4 weeks.

Even further embodiments are wherein said culture in the presence of TGFβ3 is for about 1-10 weeks, such as e.g. 1, 2, 3, 4, 5, 6, 7, 8, 9, or even 10 weeks.

In one embodiment, said culture is for about 1 week, such as 1, 2, 3, 4, 5, 6, or even 7 days.

Even further embodiments are wherein FGF2 is added in an amount of about 0.01-1 μg/ml. Further examples of amount of FGF 2 are given herein.

Still even further embodiments are wherein TGFβ3 is added in an amount of about 0.01-1 μg/ml. Further examples of amount of TGFβ3 are given herein.

Even further embodiments are wherein the mammalian MSC are human MSC.

Further embodiments are wherein the substantially homogenous population includes at least 50%, 60%, 70%, 80%, 90%, 95, 96, 07, 08, 99, 99.5, 99.9% cells expanded and differentiated with a chondrocyte phenotype.

Homogeneity of a cell population may be achieved by any method known in the art, for example, by cell sorting, e.g., flow cytometry, bead separation, or by clonal expansion.

Further embodiments are wherein the chondrocyte has a phenotype comprising expression of expression of alpha10, sox9, aggrecan, and collagen II.

In a further embodiment, said chondrocyte cell is further collagen 1 negative.

In a further embodiment, said chondrocyte cell is further collagen X negative.

In a further embodiment, said chondrocyte cell is further versecan negative.

In a further embodiment, said chondrocyte is further an integrin alpha 11 low expressing cell.

Further embodiments are wherein the MSC are selected using a solid phase technique. Examples of solid phases and of further selection processes known in the art are given in the paragraphs herein.

In one embodiment, the solid phase is a bead.

Further embodiments are wherein the MSC are selected by fluorescent cell sorting.

Further embodiments are wherein the method comprises the cell culture system according to the invention.

Culturing an alpha 10 Selected Subpopulation of MSC for Expansion and Differentiation

The MSC, such as human MSC, obtained as mentioned above are cultured in a medium which is suitable to culture the cells. Initially, the MSC are cultured in the presence of an additive promoting expansion, such as e.g. FGF2 in an amount of 1 pg/ml to 1 μg/ml, such as 0.01 to 100 ng/ml, or 0.04 to 50 ng/ml, or 0.1 to 10 ng/ml, for example, 0.5, 1, 2, 3, 4, 5, 6, 7, 8, 9, 10, 11, 12, 13, 14, 15, 16, 17, 18, 19, or even 20 ng/ml. In one embodiment, 10 ng/ml FGF2 is used upon culture. Cultivation can be carried out under conditions suitable for culturing mammal cells, usually in the presence of 5% CO, at 37° C. An example of cultivation is given below.

The mesenchymal stem cells adhering on the cultivation plate or cell flasks as mentioned above are cultured in a suitable medium as given herein in the presence of 5% CO, at 37° C. The medium is renewed every 3-4 days. FGF2 may be added from about day 5 to the medium in an amount of 10 ng/ml.

A subculture of the MSC can be performed by a suitable method known in the field of cell culture. For example, cells are collected from the plate or flasks of the primary culture which are grown close to confluence, seeded in a suitable medium containing FGF2 and cultured under the similar conditions as primary culture. When cells approach confluence, they are sub-cultured.

An example of subculture is given below. The primary culture mentioned above becomes close to confluence in around 10 days. The plate, flask or cell culture container may treated with trypsin (e.g. 0.05%)+EDTA (e.g. 0.2 mM or any other way, such as mechanical scraping, to detach the cells. Cells are collected from the plate and counted.

The cultured MSC are seeded at a density of about 5×10³ cells/cm² or any other density desired in a medium containing FGF2, e.g. 10 ng/ml, cultured and subcultured before cells become confluent. Subculture is performed by repeating the procedure of above. According to the culture method of the invention the proliferation of mesenchymal stem cells continues to 15 generations, e.g. 25 generations, over 30 generations, or for more than 16 days, such as more than 20 days, more than 30 days, more than 40 days and even more than 50 days to produce extremely high numbers of stem cells, such 1, 2, 3, 4, 5, 6, 7, 8, 9, 10, 15, 20, 25, 30, 35, 40 or even weeks.

In this culture method, FGF2 is useful as the substance which stimulates the proliferation potency of mesenchymal stem cells. Any other substance stimulating proliferation in MSC may be used. Particularly, different species may make use of different substances for stimulating proliferation and expansion of the cells. Factors like this are known and described in the art. The concentration of FGF2 in the medium is usually from 0.01 pg/ml to 1 μg/ml, such as 0.01 to 100 ng/ml, or 0.04 to 50 ng/ml, or 0.1 to 10 ng/ml, for example, 10 ng/ml. In the method of this invention any FGF, regardless of its origin, is applicable so long as it stimulates the proliferation potency of mammalian mesenchymal stem cells. FGF derived from the mammal such as FGF-1 (aFGF) or FGF-2 (bFGF) is desirable. Human FGF2 and bovine FGF2 are on the market and easily available. FGFs derived from other mammals can also be used in the invention because the receptor is common.

Cells from the above described mono layer cultures are then cultured in pellet mass cultures for differentiation chondrocytes. Examples of such cultures are given herein as well as in Johnstone et al (Exp. Cell Research 238:265-272, 1998).

### An Isolated and Substantially Homogenous Cell Population of Expanded and Differentiated Mammalian MSC

According to the invention an isolated and substantially homogenous cell population of expanded and differentiated mammalian MSC, wherein said expanded and differentiated MSC has a phenotype of a chondrocyte is disclosed.

In further embodiments, said chondrocyte phenotype comprises expression of expression of alpha10, sox9, aggrecan, and collagen II.

In a further embodiment, said chondrocyte cell is further collagen 1 negative.

In a further embodiment, said chondrocyte cell is further collagen X negative.

In a further embodiment, said chondrocyte cell is further versecan negative.

In a further embodiment, said chondrocyte is further an integrin alpha 11 low expressing cell.

Further embodiments are wherein the isolated and substantially homogenous population comprises at least 50, 60, 70, 80, 90, 95, 97, 99, 99.5, or even 99.9% expanded and differentiated MSC cells with a phenotype of a chondrocyte.

Further embodiments are wherein the cell population is obtained by the method according to the invention, or the system according to the invention.

Further embodiments are wherein the cell population is obtainable by the method according to the invention, or the system according to the invention.

### Medical Use

Further disclosed according to the invention is a cell population according to the invention, or a cell population obtained or obtainable by the method according to the invention, or a cell population obtained or obtainable by the cell culture system according to the invention, for medical use.

Furthermore, use of the cell population according to the invention, or a cell population obtained by the method according to the invention, or a cell population obtained by the cell culture system according to the invention, for the preparation of a medicament for the treatment of a cartilage condition is disclosed.

In further embodiments, said cartilage condition is encompassing any chronic or non-chronic condition, disorders, or diseases of cartilage. The term encompasses conditions including but not limited to congenital defects, meniscal injuries, damaged cartilage, degenerated cartilage, rheumatoid arthritis, osteoarthritis, cancer, congenital cartilage defect, or a traumatic or surgical injury.

As described herein, the expanded and differentiated MSC of the invention may be used to reconstitute, repopulate or repair tissue in a subject where the cells were originally isolated from that subject's own bone marrow or other tissue, i.e., autologous cells. Alternatively, the expanded and differentiated MSCs disclosed herein may be used as ubiquitous donor cells to reconstitute or repair tissue in any subject, i.e., heterologous cells.

The expanded and differentiated subpopulation of MSC with a phenotype of a chondrocyte according to the invention has broad application in treating, preventing and ameliorating any cartilage condition, e.g. disease and injury. The cells of the invention are therefore useful in many therapeutic applications including but not limited to repairing, reconstituting, repopulation and regenerating tissue as well as gene delivery and gene therapy.

The cell populations and compositions according to the invention may further be used for treatment of genetic diseases. Genetic diseases associated with a cartilage condition may be treated by genetic modification of autologous or allogeneic cells or composition is according to the invention to correct the genetic defect by introduction of a wild-type gene into the cells, either by homologous or random recombination. Methods for homologous recombination for correction of diseases are known and described by Hatada S, Nikkuni K, Bentley S A, Kirby S, Smithies O (2000, Gene correction in hematopoietic progenitor cells by homologous recombination. Proc Natl Acad Sci USA 97(25):13807-11).

With allogeneic cells, normal cells form a mammal of the same species lacking the genetic defect can be used as a therapy. Other embodiments of gene therapy may be introduction of dmg resistance genes to enable cells to have an advantage and be subject to selective pressure, e.g. the multiple drug resistance gene (MDR). More details are given in Aran J M, Pastan I, and Gottesman M M (1999, Therapeutic strategies involving the multidrug resistance phenotype: the MDR1 gene as target, chemoprotectant, and selectable marker in gene therapy. (Adv Pharmacol 46: 1-42)). Diseases where the disease is related to the lack of a particular secreted product such as a hormone, enzyme, interferon, factor, or the like, may also be treated. By employing an appropriate regulatory initiation region in or near the gene of interest, inducible production of the deficient protein may be achieved, so that production of the protein will parallel natural production, even though production will be in a different cell type from the cell type that normally produces such protein. It is also possible to insert a ribozyme, antisense or other message to inhibit particular gene products or susceptibility to diseases, particularly connective tissue

The expanded and differentiated cells of the invention may be used directly as chondrocytic cell transplants or be used in chondrocytic cell grafts either in suspension or on a cell culture support scaffold as described herein.

The expanded and differentiated cells of the invention can be placed in a carrier medium before administration. For infusion, expanded and differentiated cells of the invention can be administered in any physiologically acceptable medium, intravascularly, including intravenously, although they may also be introduced into other convenient sites such as desired site of location and/or site of target such as into joint directly or at site of cartilage condition, where the cells may find an appropriate site for regeneration. Usually, at least about 1×10⁵ cells/kg, at least about 5×10⁵ cells/kg, at least about 1×10⁶ cells/kg, at least about 2×10⁶ cells/kg, at least about 3×10⁶ cells/kg, at least about 4×10⁶ cells/kg, at least about 5×10⁶ cells/kg, at least about 6×10⁶ cells/kg, at least about 7×10⁶ cells/kg, at least about 8×10⁶ cells/kg, at least about 9×10⁶ cells/kg, at least about 10×10⁶ cells/kg, or more will be administered. See, for example, Ballen et al. (2001) Transplantation 7:635-645. The cells according to the invention may be introduced by any method including injection, catheterization, or the like. If desired, additional drugs or growth factors can be co-administered. Said additional drug or co-factor may be administered together in a single pharmaceutical composition, or in separate pharmaceutical compositions, simultaneously or sequentially with the other agents, such as drugs, either before or after administration of the other agents.

Drugs and co-factors of interest include bioactive factors anti-apoptotic agents (e.g., EPO, EPO mimetibody, TPO, mIGF-I and IGF-11, HGF, caspase inhibitors); anti-inflammatory agents (e.g., p38 MAPK inhibitors, TGF-beta inhibitors, statins, IL-6 and IL-I inhibitors, PEMIROLAST, TRANILAST, REMICADE, SIROLIMUS, and NSA/Ds (nonsteroidal anti-inflammatory drugs; e.g., TEPOXALIN, TOLMETIN, SUPROFEN); immunosupressive 1 immunomodulatory agents (e.g., calcineurin inhibitors, such as cyclosporine, tacrolimus; mTOR inhibitors (e.g., SIROLIMUS, EVEROLIMUS); anti-proliferatives (e.g., azathioprine, mycophenolate mofetil); corticosteroids (e.g., prednisolone, hydrocortisone); antibodies such as monoclonal anti-IL-2, Ralpha receptor antibodies (e.g., basiliximab, daclizumab), polyclonal anti-T-cell antibodies (e.g., antithymocyte globulin (ATG); anti-lymphocyte globulin (ALG); monoclonal anti-T cell antibody OKT3)); antithrombogenic agents (e.g., heparin, heparin derivatives, urokinase, PPack (dextrophenylalanine proline arginine chloromethylketone), antithrombin compounds, platelet receptor antagonists, anti-thrombin antibodies, anti-platelet receptor antibodies, aspirin, dipyridamole, protamine, himdin, prostaglandin inhibitors, and platelet inhibitors); and anti-oxidants (e.g., probucol, vitamin A, ascorbic acid, tocopherol, coenzyme Q-10, glutathione, L-cysteine, N-acetylcysteine) as well as local anesthetics.

Treating (or treatment of) a cartilage condition refers to ameliorating the effects of, or delaying, halting or reversing the progress of, or delaying or preventing the onset of, a cartilage condition.

An effective amount refers to a concentration of a reagent or pharmaceutical composition, such as a cell population according to the invention or other agent that is effective for producing an intended result, including cell growth and/or differentiation in vitro or in vivo, or treatment of a cartilage condition.

With respect to growth factors, differentiation factors and other additives, an effective amount may range from about 0.1 ng/ml to about 1 mg/ml.

With respect to cells according to the invention as administered to a patient in vivo, an effective amount may range from as few as several hundred or fewer to as many as several million cells or more. In specific embodiments, an effective amount may range from about 10³ to about 10¹¹ as exemplified above. It will be appreciated that the number of cells to be administered will vary depending on the specifics of the disorder to be treated, including but not limited to size or total volume/surface area to be treated, as well as proximity of the site of administration to the location of the region to be treated, among other factors familiar to the medicinal biologist.

Typically, the liquid suspension is administered in about 0.1 ml aliquots, or 0.2 ml or 0.3 ml or 0.4 ml but typically no more than 0.5 ml aliquots, at the site of injury. Typically, an aliquot, such as a 0.1 ml aliquot, contains from about 50 000 to 500 000 cells according to the invention.

The size and/or number of aliquots may vary depending on the nature and extent of the injury. The volume of the lesion (site of injury) may be accurately determined by ultrasonography. The volume of the lesion can generally be determined from the ultrasound pictures alone. Typically, when the injury is at a site which has a cavity, or can be made to form a cavity, the cavity is filled with the liquid suspension of cells (described further in e.g. Brittberg et al N Eng. J Med. 331:889-895, 1994).

An effective period (or time) and effective conditions refer to a period of time or other controllable conditions (e.g., temperature, humidity for in vitro methods), necessary or preferred for an agent or pharmaceutical composition to achieve its intended result.

Administered MSCs may also comprise a mixture of the cells according to the invention and additional cells of interest. Additional cells of interest include, without limitation, differentiated cartilage cells such as chondrocytes and any pre-stages thereof. These combinations may be useful when the expanded and differentiated cells of the invention are seeded on a three-dimensional scaffold, a hydrogel, or without any carrier.

The expanded and differentiated cells of the invention may be used to repair or reconstitute damaged or diseased tissues with a cartilage condition, such as a joint. Once the expanded and differentiated cells of the invention migrate to or are placed at the site of the condition, they may form new tissues and supplement organ function, e.g. joint function. The entire organ or part of the organ can be supplemented.

The expanded and differentiated cells of the invention may be used for implantation by contacting the cells with a tissue-engineered construct prior to grafting as noted herein. The construct containing these cells is then implanted into a host in need thereof. The cells of the invention are particularly useful for promoting cartilage generation, thereby facilitating tissue repopulation, regeneration and repair as further discussed herein.

### A Method for Reconstituting Cartilage

Also disclosed herein are methods for reconstituting, including repopulating, cartilage. As used herein reconstituting cartilage includes repopulating cartilage with new cartilage synthesizing cells as well as synthesizing new cartilage when reconstituting cartilage. Said method comprises transplanting or administering an expanded and differentiated cell population according to the invention, or a cell population obtained or obtainable by the method according to the invention, or a cell population obtained or obtainable by the cell culture system according to the invention, to a patient in the need thereof, wherein the cell population is transplanted/administered in an amount effective reconstitute cartilage tissue.

Various procedures can be contemplated for reconstituting cartilage where transferring and immobilising cells include injecting the isolated cells into the site of defect e.g. damage to articular cartilage; incubating isolated cells in suitable gel and implanting; incubating with bio-resorbable scaffold; or by systemically infusing etc. Different procedures are known in the art and described in detail by e.g. Risbud, M V and Sittenger M ((2002) Tissue Engineering: advances in in vitro cartilage regeneration. Trends in Biotech. 20(8):351-356), by Caplan, A and Bruder, S. P. ((2001) Mesenchymal stem cells: building blocks for molecular medicine in the 21st century. Trends Mol Med. 7(6):259-64), by Lazarus, H M et al ((1995) Ex vivo expansion and subsequent infusion of human bone marrow-derived stromal progenitor cells (mesenchymal progenitor cells): Implications for therapeutic use. Bone Marrow Transplant 16:557-564), and by Koc O N et al ((2000) Rapid hematopoietic recovery after coinfusion of autologousblood stem cells and culture-expanded marrow mesenchymal stem cells in advanced breast cancer patients receiving high-dose chemotherapy. J. Clin. Oncol 18(2):307-516).

Thus, a further embodiments are wherein said cells are reconstituted using a scaffold, such as a bio-resorbable, bio-compatible scaffold known in the art.

Optionally the cells can be incubated with an antibody to the integrin alpha10 in order to hold the cells in place. Thus, antibodies can be conjugated to a bio-resorbable scaffold allowing immobilization of the cells before implantation into the damaged or defect site.

The shape and dimensions of the 3-D scaffold are determined based on the organ being replaced or supplemented, and the type of scaffold material being used to create the construct. For example, if a polymeric scaffold is used, the dimension of the polymeric scaffold may vary in terms of width and length of the polymeric scaffold. One skill in the art recognizes that the size and dimensions of the polymeric scaffold will be determined based on the area of the organ being replaced or supplemented.

A scaffold allows 3D-immobilization of cells. Suitable biomaterial scaffolds are exemplified below. The examples given are not limiting the use of other suitable scaffolds obvious to a skilled artisan to choose if more suitable for the particular application. Types of scaffold further include, bioresorbable poly(a-hydroxy esters) scaffolds such as polylactic acid (PLLA), polyglycolic acid (PGA) and copolymer (PLGA). Further embodiments include scaffolds derived from polymeric gels such as hyaluronic acid, collagen, alginate and chitosan known in the art.

Further embodiments include scaffolds derived from porous carriers, such as tricalcium phosphate and/or hydroxyapatite ceramic block (see Luyten, F. P, DellYAccio, F and De Bari, C (2001) Skeletal tissue engineering: opportunities and challenges, Best Prac & Res. Clin. Rheum. 15(5):759-770).

The cells of the invention may be surgically implanted, injected, delivered, e.g., by way of a catheter or syringe, or otherwise administered directly or indirectly to the desired site of localization or targeting, i.e. site in need of repair or augmentation, as exemplified in other paragraphs herein. The cells may further be administered by way of a matrix, e.g., a three-dimensional scaffold as mentioned previously. The cells may be administered with conventional pharmaceutically acceptable carriers. Examples of suitable carriers are given in other paragraphs herein.

Routes of administration of the cells of the invention or compositions or components (including e.g., other cells, ECM, cell lysate, conditioned medium) thereof include intramuscular, ophthalmic, parenteral (including intravenous), intraarterial, subcutaneous, oral, and nasal administration. Particular routes of parenteral administration include, but are not limited to, intramuscular, subcutaneous, intraperitoneal, intracerebral, intraventricular, intra-cerebroventricular, intrathecal, intracistemal, intraspinal and/or peri-spinal routes of administration.

When cells are administered in semi-solid or solid devices, surgical implantation into a precise location in the body is typically a suitable means of administration. Liquid or fluid pharmaceutical compositions, however, may be administered to a more general location (e.g., throughout a diffusely affected area, for example), from which they migrate to a particular location, i.e. a location of a cartilage condition, by e.g., responding to chemical signals such as chemotactic signals or other cellular signals affecting migration and tissue/organ invasion.

### A Method of Treating a Cartilage Condition

Also disclosed herein is a method of treating a cartilage condition. Said method comprises transplanting an isolated and substantially homogenous cell population of expanded and differentiated mammalian MSC according to the invention for reconstitution of cartilage wherein said MSC has a phenotype of a chondrocyte. Said method further comprises administering an expanded and differentiated cell population according to the invention, or a cell population obtained or obtainable by the method according to the invention, or a cell population obtained or obtainable by the cell culture system according to the invention, to a patient in the need thereof, wherein the cell population is administered in an amount effective to reconstitute cartilage tissue and to treat said cartilage condition.

Examples of cartilage conditions are given throughout the detailed description of the invention and include, but are not limited to, damaged cartilage, degenerated cartilage, rheumatoid arthritis, osteoarthritis, trauma, cancer, congenital cartilage defect, or a traumatic or surgical injury.

Further embodiments are wherein said expanded and differentiated cell population according to the invention, or a cell population obtained or obtainable by the method according to the invention, or a cell population obtained or obtainable by the cell culture system according to the invention are administered using a scaffold. Examples of suitable scaffolds are given in paragraphs both above and below.

Dosage forms and regimes for administering cells and compositions according to the invention or any of the pharmaceutical compositions described herein are developed in accordance with good medical practice, taking into account the condition of the individual patient, e.g., nature and extent of the condition being treated, age, sex, body weight and general medical condition, and other factors known to medical practitioners. Thus, the effective amount of a pharmaceutical composition to be administered to a patient is determined by these considerations as known in the art. Further examples of dosage forms and regimens are given in the paragraphs herein.

### A Pharmaceutical Composition

According to the invention a pharmaceutical composition is disclosed. Said composition comprises an expanded and differentiated cell population according to the invention, or a cell population obtained or obtainable by the method according to the invention, or a cell population obtained or obtainable by the cell culture system according to the invention and a pharmaceutical acceptable carrier or excipient.

A pharmaceutically acceptable carrier (or medium), may be used interchangeably with the term biologically compatible carrier or medium and refers to reagents, cells, compounds, materials, compositions, and/or dosage forms which are, within the scope of sound medical judgment, suitable for use in contact with the tissues of mammals such as human beings and animals without excessive toxicity, irritation, allergic response, or other complication commensurate with a reasonable benefit/risk ratio. Examples of pharmaceutically acceptable carriers suitable for use in the present invention include liquids, semi-solid (e.g., gels) and solid materials (e.g., scaffolds). Further examples of pharmaceutically acceptable carriers for the cells of the invention include organic or inorganic carrier substances suitable which do not deleteriously react with the cells of the invention or compositions or components thereof. To the extent they are biocompatible, suitable pharmaceutically acceptable carriers include water, salt solution (such as Ringer's solution), alcohols, oils, gelatins, and carbohydrates, such as lactose, amylose, or starch, fatty acid esters, hydroxymethyl cellulose, and polyvinyl pyrolidine. Such preparations can be sterilized, and if desired, mixed with auxiliary agents such as lubricants, preservatives, stabilizers, wetting agents, emulsifiers, salts for influencing osmotic pressure, buffers, and colouring. Pharmaceutical carriers suitable for use in the present invention are known in the art and are described, for example, in Pharmaceutical Sciences (17^{th} Ed., Mack Pub. Co., Easton, Pa.) and WO 96105309, each of which are incorporated by reference herein.

The term biodegradable describes the ability of a material to be broken down (e.g., degraded, eroded, dissolved) in vivo. The term includes degradation in vivo with or without elimination (e.g., by resorption) from the body. The semi-solid and solid materials may be designed to resist degradation within the body (non-biodegradable) or they may be designed to degrade within the body (biodegradable, bioerodable). A biodegradable material may further be bioresorbable or bioabsorbable, i.e., it may be dissolved and absorbed into bodily fluids (water-soluble implants are one example), or degraded and ultimately eliminated from the body, either by conversion into other materials or by breakdown and elimination through natural pathways.

Various procedures can be contemplated for transferring and immobilising said pharmaceutical composition including injecting the isolated cells into the site of defect e.g. damage to articular cartilage; incubating isolated cells in suitable gel and implanting; incubating with bio-resorbable scaffold; or by systemically infusing etc. Different procedures are known in the art and described in detail by e.g. Risbud, M V and Sittenger M ((2002) Tissue Engineering: advances in in vitro cartilage regeneration. Trends in Biotech. 20(8):351-356), by Caplan, A and Bruder, S. P. ((2001) Mesenchymal stem cells: building blocks for molecular medicine in the 21st century. Trends Mol Med. 7(6):259-64), by Lazarus, H M et al ((1995) Ex vivo expansion and subsequent infusion of human bone marrow-derived stromal progenitor cells (mesenchymal progenitor cells): Implications for therapeutic use. Bone Marrow Transplant 16:557-564), and by Koc O N et al ((2000) Rapid hematopoietic recovery after coinfusion of autologousblood stem cells and culture-expanded marrow mesenchymal stem cells in advanced breast cancer patients receiving high-dose chemotherapy. J. Clin. Oncol 18(2):307-516).

Thus, a further embodiment of the pharmaceutical composition comprises a scaffold, such as a bio-resorbable, bio-compatible scaffold known in the art. A review of scaffold design is provided by Hutmacher, J. Biomat. Sci. Polymer Edn., 12(1):107-124 (2001). Further examples are given on other paragraphs herein.

Optionally the cells can be incubated with an antibody, e.g. to the integrin alpha10, in order to hold the cells in place. Thus, antibodies can be conjugated to a bio-resorbable scaffold allowing immobilization of the cells before implantation into the damaged or defect site.

The scaffold allows 3D-immobilization of cells. Suitable biomaterial scaffolds are exemplified above and below. The examples given are not limiting the use of other suitable scaffolds obvious to a skilled artisan to choose if more suitable for the particular application. Types of scaffold include, bioresorbable poly(a-hydroxy esters) scaffolds such as polylactic acid (PLLA), polyglycolic acid (PGA) and copolymer (PLGA). Further embodiments include scaffolds derived from polymeric gels such as hyaluronic acid, collagen, alginate and chitosan, as discussed above.

Further embodiments include scaffolds derived from porous carriers, such as tricalcium phosphate and/or hydroxyapatite ceramic block (Luyten, F. P, DellYAccio, F and De Bari, C (2001) Skeletal tissue engineering: opportunities and challenges. Best Prac & Res. Clin. Rheum. 15(5):759-770).

### Medical Need

A patient in the need thereof may be any mammal in the need of cartilage reconstitution, repopulation or cartilage repair due to a cartilage condition. Said cartilage condition may be any damaged cartilage, degenerated cartilage, rheumatoid arthritis, osteoarthritis, trauma, cancer, congenital cartilage defect, or a traumatic or surgical injury.

The term patient or subject refers to animals, including mammals, preferably humans, who are treated with the cells according to the invention or a pharmaceutical composition according to the invention in accordance with the methods described herein.

Examples of mammals are humans, rats, dogs, mice, horses, cats, cows, sheep, goats, and camels.

In one embodiment, the mammal is a patient in the need thereof being a human in the need thereof.

### Kits According to the Invention

Kits, such as e.g. kit of parts, are also encompassed and disclosed in the present invention.

Thus, a kit for expanding and differentiating isolated mammalian MSC to a chondrocyte comprising the culture system according to the invention is disclosed.

Further embodiments are wherein the kit further comprises instructions to culture said isolated MSC using the methods according to the invention.

Even further, a kit comprising an expanded and differentiated cell population according to the invention, or a cell population obtained or obtainable by the method according to the invention, or a cell population obtained or obtainable by the cell culture system according to the invention.

Further embodiments are wherein said kit further comprising means for delivering the cell population to a patient in the need thereof.

Means for delivering said cell population may be any means for transferring or transferring and immobilising said cell population including injecting the isolated cells into the site of defect e.g. damage to articular cartilage; incubating isolated cells in suitable gel and implanting; incubating with bio-resorbable scaffold; or by systemically infusing etc. Different procedures are known in the art and described in detail by e.g. Risbud, M V and Sittenger M ((2002) Tissue Engineering: advances in in vitro cartilage regeneration. Trends in Biotech. 20(8):351-356), by Caplan, A and Bruder, S. P. ((2001) Mesenchymal stem cells: building blocks for molecular medicine in the 21st century. Trends Mol Med. 7(6):259-64), by Lazarus, H M et al ((1995) Ex vivo expansion and subsequent infusion of human bone marrow-derived stromal progenitor cells (mesenchymal progenitor cells): Implications for therapeutic use. Bone Marrow Transplant 16:557-564), and by Koc O N et al ((2000) Rapid hematopoietic recovery after coinfusion of autologousblood stem cells and culture-expanded marrow mesenchymal stem cells in advanced breast cancer patients receiving high-dose chemotherapy. J. Clin. Oncol 18(2):307-516).

Thus, a further embodiment of the kit comprises a scaffold, such as a bio-resorbable, bio-compatible scaffold known in the art. Examples of scaffolds are given in detail in the paragraphs throughout the detailed description herein.

Even further embodiments of the kit comprises means for determining that the delivered cell population locate to at least one desired site, e.g. site of cartilage condition to locate and/or target. Such means should allow for localization, detection, enumeration or even quantification of delivered cells to said patient.

Survival, as well as localization, detection, enumeration, quantification of administered, i.e. delivered, cells in a living patient and analysis of the degree cell engraftment or reconstitution may be determined through the use of a variety of scanning techniques, e.g., computerized axial tomography (CAT or CT) scan, magnetic resonance imaging (MRI) or positron emission tomography (PET) scans. Determination of cell transplant localization and survival can also be done post mortem by removing the target tissue, and examining it visually or through a microscope. Alternatively, cells can be treated with stains that are specific for cells of a specific lineage. Transplanted cells can also be identified by prior incorporation of tracer dyes such as rhodamine- or fluorescein-labeled microspheres, fast blue, bisbenzamide, ferric microparticles, or genetically introduced reporter gene products, such as beta-galactosidase or beta-glucuronidase. A further method is to radiolabel the cells with tritiated thymidine ([³H-thymidine]) before delivery to the patient to be able to detect said cells after delivery or implantation. After implantation, the degree of radioactivity in a tissue can be correlated to cell engraftment in a linear fashion. Furthermore, functional integration of transplanted cells according to the invention into a subject can be assessed by examining restoration of the function that was damaged or diseased, for example, restoration of joint, or augmentation of function.

Examples of cartilage conditions are given in the paragraphs herein and include, but are not limited to site of cartilage injury and site of cartilage repair.

In a further embodiment, at least one desired site is 1, 2, 3, 4, 5, 6, 7, 8, 9, 10 or even more desired sites, such as 10, 20, 30, 40 50, or more, desired sites, depending on the nature of the cartilage condition to target and/or locate.

In further embodiments, the delivered cell population results in the same physiological response as a normal organ. This physiological response may then, of course, be tested and analysed.

In a further embodiment the desired site is cartilage.

In further embodiments, the desired site is articular cartilage.

Also disclosed is a kit for reconstitution of cartilage. Said kit comprises
a) an expanded and differentiated cell population according to the invention, or a cell population obtained by the method according to the invention, or a cell population obtained by the cell culture system according to the invention,
b) means for reconstituting cartilage, and
c) optionally instructions for reconstituting cartilage.

In further embodiments, means for reconstituting cartilage is as given in paragraphs herein.

Further embodiments of the kit include additional components, such as a matrix (e.g., a scaffold), hydrating agents (e.g., physiologically-compatible saline solutions, prepared cell culture media), cell culture substrates (e.g., culture dishes, plates, vials, etc.), cell culture media (whether in liquid or powdered form), antibiotic compounds, hormones, additives for e.g. expansion and differentiation of the cell population, and the like. While the kit can include any such components, preferably it includes all ingredients necessary for its intended use. If desired, the kit also can include cells, typically cryo-preserved, according to the invention. Included cells may also be isolated mammalian MSC. Said cells, both according to the invention and others, may be seeded into the scaffolds as described herein.

Further embodiments of the kits include cells according to the invention, components and products of the cells in various methods for augmentation, regeneration, and repair as described above. In some embodiments, the kits may include one or more cell populations, including at least the cells according to the invention and a pharmaceutically acceptable carrier (liquid, semi-solid or solid). The kits also optionally may include a means of administering the cells, for example by injection. The kits further may include instructions for use of the cells. Kits prepared may further include full procedure supplies including tissue scaffolds, surgical sutures, and the like, where the cells are to be used in conjunction with repair of acute injuries. Kits for assays and in vitro methods may contain one or more of (1) cells or cell populations of the invention, (2) reagents for practicing the in vitro method, (3) other cells or cell populations, as appropriate, and (4) instructions for conducting the in vitro method.

Further embodiments of the kits according to the invention may comprise other suitable articles such as a scaffold, a cell culture support surface, medical devices, extracorporeal devices and artificial joints, tubes, sutures, stents, orthopedic devices, vascular grafts, membranes, films, biosensors, or microparticles.

### Subpopulation of MSC

Also included in the present invention is the use of alpha10 as a marker for a subpopulation of MSC with enhanced chondrogenic potential. Further details are given herein for such use and also in the accompanying examples.

Furthermore, a method of identifying a subpopulation of MSC with enhanced chondrogenic potential is disclosed. The method comprises
a) isolating a population of cells comprising MSC,
b) detecting integrin alpha10 expression on a subpopulation of said MSC,
c) comparing the alpha10 expression to a control cell population not expressing alpha10,
d) identifying said alpha10 expressing cells as subpopulation of MSC with an enhanced chondrogenic potential.
   Isolation a population of MSC and detection of alpha10 expression is described herein. Comparing the alpha10 expression to a cell population not expressing alpha10 is done either via an alpha10 negative fraction of cells isolated in parallel with the alpha10 expressing cells, or by comparing the expression to cells transfected with the alpha10-gene thereby expressing the protein. Cells expressing the alpha10 integrin are thus identified as a subpopulation of MSC. Said subpopulation is shown herein to have an enhanced chondrogenic potential, which could be readily tested by methods disclosed herein.

Further embodiments are wherein the detection of alpha10 is done by immunological means.

Still further embodiments are wherein the immunological means comprises adding an antibody specifically reacting with alpha10. Examples of such antibodies are given herein. Analysis of the protein alpha10 expression may be performed by e.g. FACS analysis as described herein, or by any other immunological means known in the art such as cell-ELISA (Enzyme linked immunosorbent assay), or Western blot. Protocols for such assays are available in the art and may be found in e.g. Antibodies—A Laboratory Manual (Harlow & Lane, Cold Spring Harbor Laboratory, 1988, ISBN0 0-87969314-2)

Further embodiments include wherein the detection of alpha10 is done by identifying expression of the alpha10-gene. The expression of the gene may be identified by e.g. RT-PCT, as described and exemplified herein, as well as by probing the alpha10 mRNA transcripts in a fraction of the expected alpha10-expressing cells using e.g. a Northern blot or similar techniques know in the art (see e.g. (see Sambrook & Russell, 2000, Molecular Cloning, A Laboratory Manual, Third Edition, Cold Spring Harbor, N.Y.).

While the present invention has been particularly shown and described with reference to the presently disclosed embodiments, it is understood that the invention is not limited to the embodiments specifically disclosed and exemplified herein. Numerous changes and modifications may be made to the preferred embodiment of the invention, and such changes and modifications may be made without departing from the scope and spirit of the invention as set forth in the appended claims.

Various patents and other publications are cited herein and throughout the specification, each of which is incorporated by reference herein in its entirety.

### EXAMPLES

### Example 1

### Isolation of Mesenchymal Stem Cells from Human Bone Marrow

### Objective

The objective with this example was to demonstrate that certain growth factors could affect the mRNA expression of integrin alpha10 on human MSC.

### Materials and Methods

A. Isolation of Mesenchymal Stem Cells from Human Bone Marrow

Posterior iliac aspirations were performed on healthy volunteers for adult bone marrow (BM) collection. The human bone marrow cells were diluted in equal amount of PBS (with Ca2+ and Mg2+) (GibcoBRL, Paisley, UK), 0.6% NaCitrate (Sigma, Sweden), 0.1% BSA (SERVA Electrophoresis GmbH, Heidelberg, Germany) and 100 U/ml DNase (Sigma, Sweden).

Mononuclear cells (MNCs) were isolated by layering the bone marrow cells on a density gradient (Lymphoprep™, density 1.077 g/ml, Nycomed, Norway) accordingly to the manufactures descriptions.

MNC were washed twice in PBS and resuspended in MEM α-Medium (GibcoBRL, Paisley, UK) with 20% FCS, 100 U/ml Penicillin and 100 μg/ml streptomycin (GibcoBRL, Paisley, UK) and 1× Glutamax (GibcoBRL, Paisley, UK) and cultures at a density of 0.8×10⁶ cells/cm².

After 4 days of culture non adherent cells were removed by changing medium. Every 3-4 days the medium was changed and the cells were cultured until sub-confluence and passage at a density of about 5000 cells/cm².

The bone marrow derived MSCs were cultured for 4 weeks before stimulation and stimulated for 5 days with different growth factors. 5×10⁴ MSCs cells/well were stimulated in 6 wells plate with 10 ng/ml FGF2 (BioSource Europe SA, Belgium), 10 ng/ml TGFβ3 (R&D Systems Europe Ltd., United Kingdom), 100 ng/ml BMP2 (R&D Systems Europe Ltd., United Kingdom), 100 ng/ml BMP7 (R&D Systems Europe Ltd., United Kingdom) and 100 ng/ml IGF1 (R&D Systems Europe Ltd., United Kingdom) in MEM a-Medium (GibcoBRL, Paisley, UK) 20% FCS, 100 U/ml Penicillin and 100 μg/ml streptomycin (GibcoBRL, Paisley, UK) and 1× Glutamax (GibcoBRL, Paisley, UK). Growth factors were added at day 1 and 3 during the 5 days stimulation.

Total RNA was isolated with Qiagen RNeasy (QIAGEN, GmbH, Germany) according to the manufactures protocol. Total RNA (1 μg) was reverse transcribed by Superscript II (200 units) (Invitrogen™ Life Technologies, Carlsbad, Calif.) using random hexamer oligonucleotides. The quantitative PCR was performed using the LightCycler® FastStart DNA Master SYBR GreenI (Roche Applied Science, Mannheim, Germany). All PCRs were performed at a thermal profile of 95° C. for 10 s, 65° C. 5 s, 72° C. 15 s.

TABLE 2
Primers used

| | | |
|---|---|---|
| Primers | sequence | length |

| | | |
|---|---|---|
| GAPDH | F AACAGCGACACCCACTCCTC | 341 |
| | R GGAGGGGAGATTCAGTGTGTGGT | |
| COL1A1 | F GCTTCCCTGGTCTTCCTG | 187 |
| | R TCTCACCACGGTCACCCT | |
| COL2A1 | F GTTATCGAGTACCGGTCACAGAAG | 174 |
| | R AGTACTTGGGTCCTTTGGGTTTG | |
| α1 | F TCAGCCAAGTCAATGTTTCG | 197 |
| | R GACCCATAATGGCACTCTGC | |
| α2 | F CGGGTGTGTGTTCTGACATC | 201 |
| | R ACCCCACCTGTGTCTTTGTG | |
| α10 | F TCTCTAGAAACCTCCACCTGG | 438 |
| | R CTGGAAGGAGGGCTGAGATGATGA | |
| α11 | F GCTGCAGGCAGTGACAGTA | 254 |
| | R GCGATGGGAATGGTGATCT | |
| Aggrecan | F CAGCACCAGCATCCCAGA | 167 |
| | R CAGCAGTTGATTCTGATTCACG | |
| Sox9 | F GAGAACACGTTCCCCAAGG | 200 |
| | R CGTTCTTCACCGACTTCCTC | |
| Versican | F AGATGGGTTCATGGGTAATT | 189 |
| | R CTATACGTGCAAGAAAGGAACAGT | |

### Results

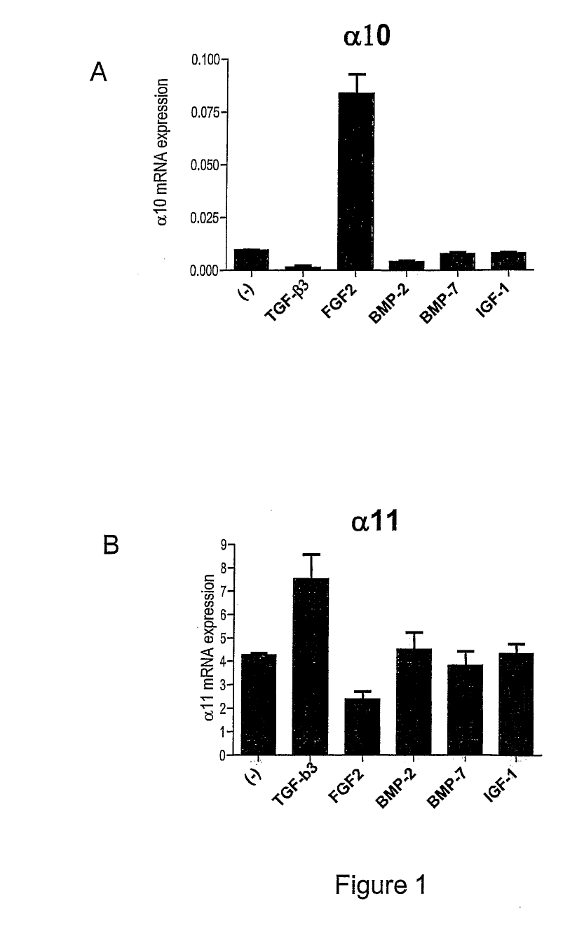

Human mesenchymal progenitor cells (HMSC) were cultured for five days in the presence of TGF-β3, FGF-2, BMP-2, BMP-7 or IGF-1 and analysed for integrin alpha10 and alpha11 mRNA expression. Human MSCs cultured in monolayer for five days in the presence of FGF-2 had an 8-fold increase in mRNA expression for integrin alpha10 compared to un-treated cells (FIG. 1A). The FGF-2-treatment of hMSCs also resulted in a decreased mRNA expression for integrin alpha11 (FIG. 1B). TGF-B3 treatment of monolayer hMSC for five days resulted in a decreased integrin alpha10 (FIG. 1A) and an increased alpha11 (FIG. 1B) mRNA expression. Neither, BMP-2, BMP-7 or IGF-1 treatment resulted in any regulation of integrin alpha10 or alpha11 mRNA expression under these conditions.

### Conclusion

It is here demonstrate that FGF-2 treatment during monolayer culture results in a population of hMSCs that has an increased expression of integrin alpha10 and a decreased expression of integrin alpha11.

### Example 2

### Regulation of Alpha 10 and Alpha 11

### Objective

The objective with this example was to demonstrate that FGF2 regulates the cell-surface expression of integrin alpha10 and alpha11 in hMSC

### Materials and Methods

Human MSCs were isolated and cultured as described in Example 1. The primary antibodies used were mAb365 mIgG₂ₐ (anti-alpha 10), with isotype control IgG₂ₐ, C09-biotin (anti-alpha11) and the isotype control CT17-biotin at a concentration of 1 μg/ml (both antibodies from Bioinvent Int AB, Sweden).

Secondary antibodies used were Cy™5 conjugated anti-mIgG (Jackson ImmunoResearch, Pennsylvania) and PE conjugated Streptavidin (BD, San Jose, Calif.). The FACS staining was done according to the manufacturer's instructions. The cell marker expression was detected with a FACSort (13D, San Jose, Calif.) and analyzed using the CellQuest® software (BD, San Jose, Calif.).

### Results

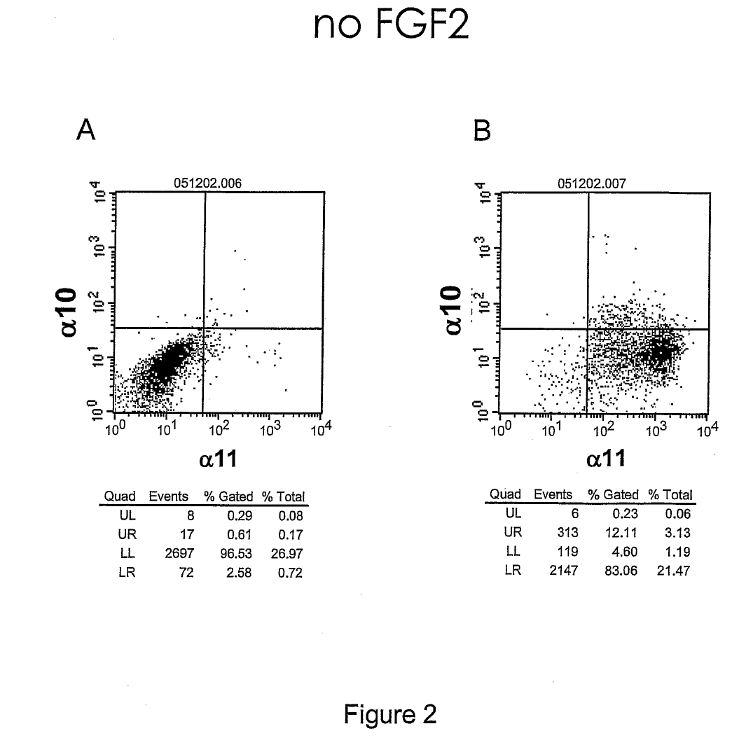

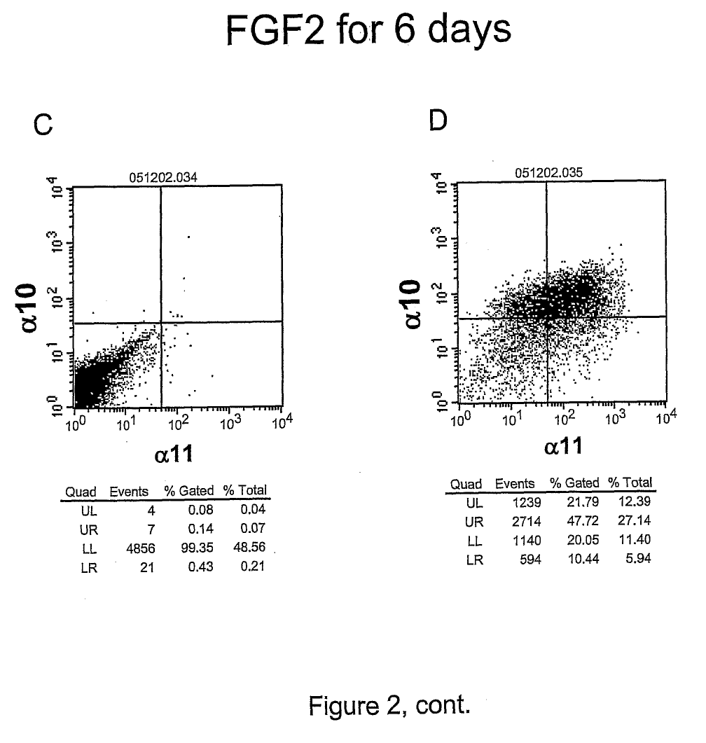

FGF2 treatment of hMSC for 6 days resulted in an increase from 12% to 70% of alpha10 positive cells (FIG. 2). The percentage alpha11 positive hMSC decreased from 95% to 58%.

### Conclusion

The results demonstrate that a hMSC population treated with FGF2 has an altered cell-surface expression of integrins were the phenotype has shifted towards the alpha10 expressing chondrocyte phenotype.

### Example 3

### Human MSC with an Enhanced Chondrocyte Potential

### Objective

The objective with this example was to demonstrate that integrin alpha10-high/alpha11-low hMCSs has an enhanced chondrogenic potential compared to integrin alpha10-low/alpha11-high hMCSs

### Materials and Methods

Human MSCs were isolated as described in example 1. At day 7 after isolation MSCs were cultured in presence or absence of 10 ng/ml FGF2 (BioSource Europe SA, Belgium) for 14 days. The cells were stained and FACS-analyzed as described in example 2.

MSCs were induced to chondrogenic phenotype in pellet mass culture using 2×10⁵ cells/pellet in DMEM (GibcoBRL, Paisley, UK) supplemented with 1× Insulin-transferrin sodium selenite (Sigma, Sweden), 0.1 μM dexamethasone (Sigma, Sweden), 50 μM ascorbic acid (Sigma, Sweden), 1 mg/ml Linoleic acid-bovine serum albumin (Sigma, Sweden), 1% Nonessential AA (GibcoBRL, Paisley, UK), 100 U/ml Penicillin, 100 μg/ml Streptomycin (GibcoBRL, Paisley, UK) and 10 ng/ml TGF-β3 (R&D Systems Europe Ltd., United Kingdom).

To determine the chondrogenic differentiation the pellet cultures were tested for Collagen type II, α10-, α11-integrins, Sox 9 and aggrecane expression at mRNA level.

Total RNA was isolated with Qiagen RNeasy (QIAGEN, GmbH, Germany) according to the manufactures protocol. Total RNA (1 μg) was reverse transcribed by Superscript II (200 units) Invitrogen™ Life Technologies, Carlsbad, Calif.) using random hexamer oligonucleotides.

Quantitative PCR was performed using the LightCycler® FastStart DNA Master SYBR GreenI (Roche Applied Science, Mannheim, Germany). All PCRs were performed at a thermal profile of 95° C. for 10 s, 65° C. 5 s, 72° C. 15 s.

Newly synthesized collagen II expression was also measured at protein level with a procollagen II ELISA (IBEX Technologies Inc., Montreal, Quebec, Canada) according to the manufactures description.

Proteoglycan synthesis in pellet mass culture was measured by metabolic labelling with S³⁵. In brief, pellets where pulsed with 50 μCi/ml ³⁵S for 4 hours, washed with 200 μl PBS and digested over night with 10 U Papain (SIGMA #P3125) in 200 μl 100 mM NaAc, 10 mM Cysteine-hydrochloride, 2 mM EDTA, pH ˜5.5.

Free isotope was removed by precipitation of proteoglycan with hexadecyl pyridinium chloride monohydrate (SIGMA# C5460) at a final concentration of 30 mM in the presence of 100 μg/ml chondroitin sulphate-6 (SIGMA# C4384). The precipitate was collected by centrifugation at 5000×g for 10 minutes and subsequently washed two times with precipitation buffer before it was finally dissolved in concentrated formic acid and counted in a β-counter.

### Results

Human MSCs, cultured with or without FGF2, were subjected to chondrocyte differentiation in pellet-mass. Before differentiation the cells pre-treated with FGF2 were verified as being integrin alpha10-high/alpha11-low and the hMSCs not treated with FGF2 were verified as integrin alpha10-low/alpha11-high using FACS analyses (see example 2).

Total-RNA was extracted from pellets on day 7, day 14 and day 21 after onset of pellet-mass and quantitatively analyzed for gene-expression of GAPDH, collagen type II (COL2A), aggrecan, integrin alpha10 (ITGA10), integrin alpha11 (ITGA11) and SOX9.

Supernatant was collected and analyzed for newly synthesized collagen type II (CPII pro-peptide) and separate pellets were analyzed for proteoglycan synthesis using 35-S incorporation.

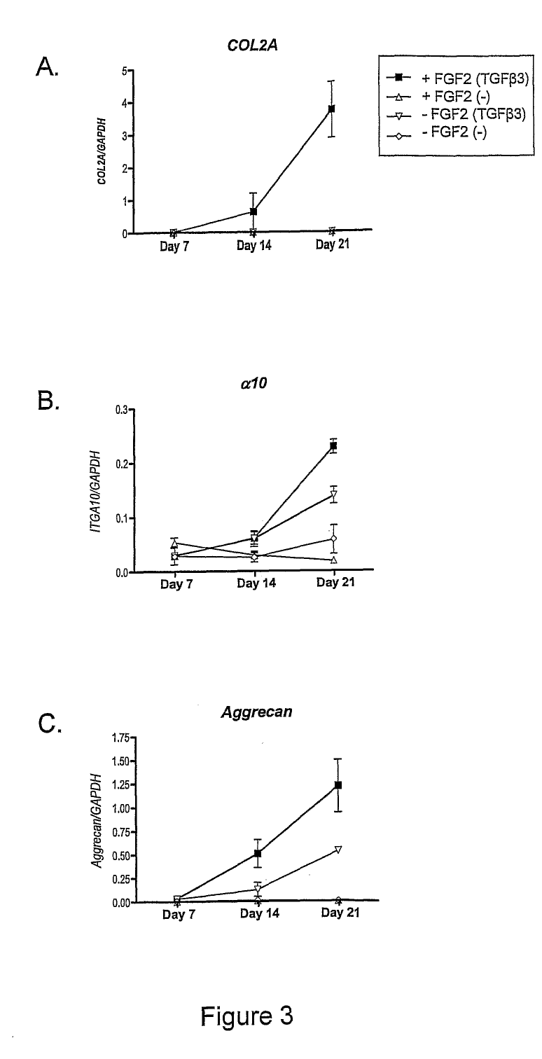

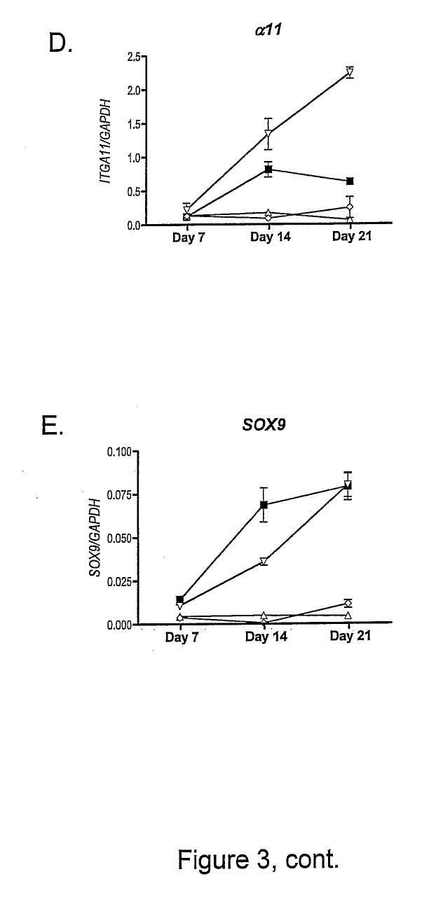

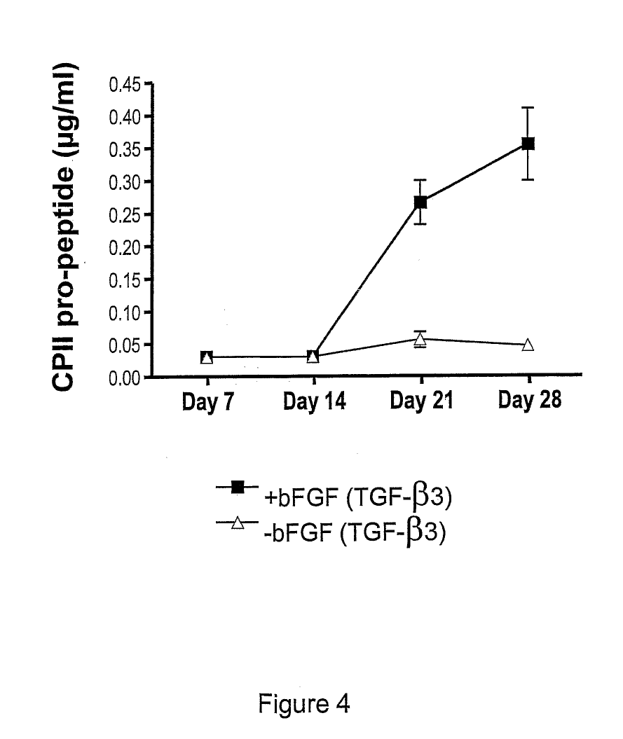

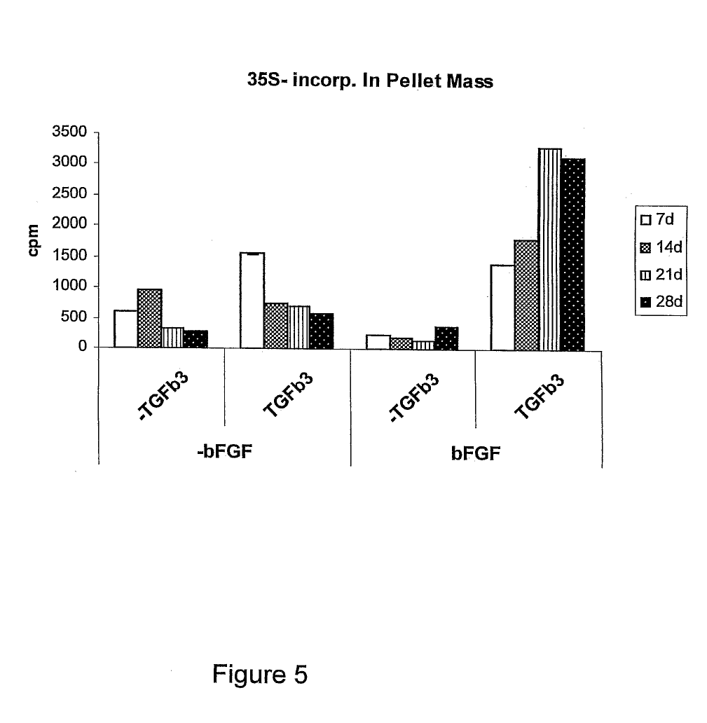

The results show that the FGF2 pre-treated cells has an increased mRNA expression of COL2A, aggrecan, SOX9 and ITGA10 compared to the un-treated cells (FIG. 3A). The expression increases over time reaching maximum levels at day 21. The ITGA11 mRNA level is highest in the pellets originating from hMSC that has not been pre-treated with FGF2. The data also clearly demonstrate that the cartilage formation is completely dependent on TGF-B since omitting TGF-B from the pellet-cultures results in low-level expression of the chondrocyte markers COL2A, SOX9, aggrecan and ITGA10.

By analyzing the supernatans from the pellet-cultures for newly synthesized collagen type II protein (i.e. CPII pro-peptide), it was verified that the FGF2 pre-treated hMSCs synthesize and process collagen type II (FIG. 3B). The pellet cultures from un-treated hMSCs did not synthesize detectable levels of CPII pro-peptide.

Proteoglycan synthesis (i.e. 35-S incorporation) was also analyzed at different time-points during pellet formation. The results show that the FGF2 pre-treated hMSCs have an increased proteoglycan synthesis compared to the un-treated cells and that the proteoglycan synthesis peaks around day 21 (FIG. 3C). The proteoglycan synthesis is clearly TGF-13 dependent.

### Conclusion

It is concluded that a human mesenkymal stem cell population cultured under conditions that favours the integrin alpha10-high/alpha11-low phenotype, has an enhanced capacity to synthesize cartilage under chondrocyte differentiation conditions compared to a stem cell population having the integrin alpha10-low/alpha11-high phenotype.

### Example 4

### Enrichment of Alpha 10 Expressing Cells from BM

### Objective

The objective with this example was to demonstrate that by using integrin alpha10 specific monoclonal antibodies it is possible to enrich human mesenchymal progenitor cells (IMSC) directly from bone marrow derived mononuclear cells (MNC).

### Materials and Methods

Posterior iliac aspirations were performed on healthy volunteers for adult bone marrow (BM) collection. The human bone marrow cells were diluted in equal amount of PBS (with Ca2+ and Mg2+) (GibcoBRL, Paisley, UK), 0.6% NaCitrate (Sigma, Sweden), 0.1% BSA (SERVA Electrophoresis GmbH, Heidelberg, Germany) and 100 U/ml DNase (Sigma, Sweden).

Mononuclear cells (MNCs) were isolated by layering the bone marrow cells on a density gradient (Lymphoprep™, density 1.077 g/ml, Nycomed, Norway) accordingly to the manufactures descriptions.

Human MNCs were labelled with 10 μg/ml mAb365-biotin, (alpha 10 integrin antibody) or the isotype control mIgG2a-biotin. The labelled cells were incubated for 20 minutes at 4° C., washed and incubated with anti-biotin MicroBeads (MitenyiBiotec, Germany) for 20 minutes in 4° C.

Alpha 10 positive cells were isolated by positive selection with an LS midiMACS column (MitenyiBiotec, Germany) according to the manufactures descriptions.

Total bone marrow, the positive and negative fraction were seeded at 1000 cells/well in 96 wells plates.

After 8 or/and 12 days of culture, wells with proliferative clones of fibroblast shaped cells were counted. Clones from the total bone marrow and from the mAb365 isolated cells were further cultured in presence or absence of 10 ng/ml FGF2 (BioSource Europe SA, Belgium) and analyses for α10 and α11 expression by FACS.

### Results

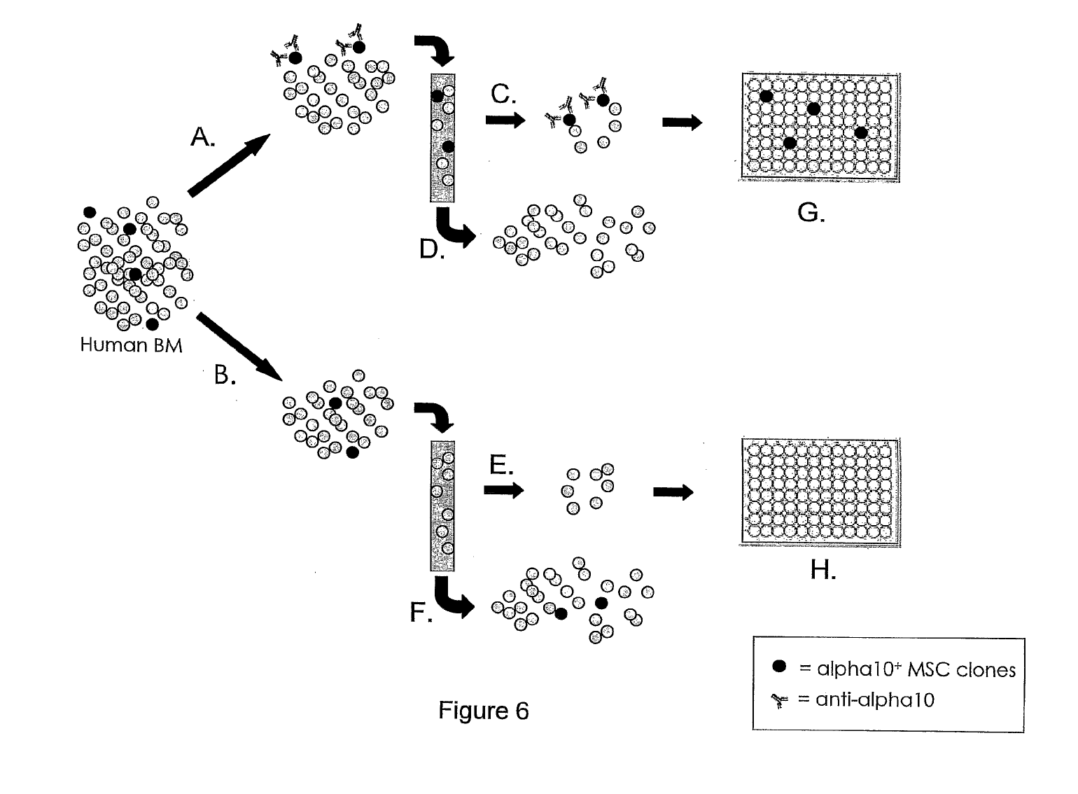

FIG. 6 shows an outline of the cell separation protocol. In FIG. 6 human BM cells are incubated either with integrin alpha10 antibodies (A) or an isotype control (B). Fraction C is eluted from the column as alpha 10 selected cells. Fraction D is the negative fraction, not binding to alpha 10 antibodies. Similarly, fraction E corresponds to cells that bind to isotype control and fraction F the negative fraction not binding to isotype control, merely passing through the column. All four fractions are seeded into separate 96 well plates (G and H). Fraction C is enriched for a subpopulation of MSC with an enhanced capacity to differentiate to chondrocyte cells.

Mononuclear cells from human bone marrow samples were subjected to magnetic bead-based separation using a monoclonal antibody specific for integrin alpha10 (mAb365).

Negative controls were identical separations where the integrin alpha10 antibody had been excluded.

After separation the positive cell-fraction containing integrin alpha10 positive cells was seeded into 96-well plates (1000 cells/well). On day 4, 6 and 8 after plating the wells were monitored for proliferating cells with a fibroblastic phenotype. On day 8, 24% of the wells containing cells subjected to mAb365 separation contained proliferating cells with an hMSC appearance. Excluding the integrin alpha 10 antibody did not result in enrichment of proliferating hMSCs.

### DISCUSSION/CONCLUSION

It is concluded that by using antibodies specific for integrin alpha10 it is possible to enrich proliferating cells with an hMSC appearance directly from human bone-marrow.

### Example 5

### Chondrogen Differentiation of Alpha 10 Isolated Cells

### Objective

The objective with this example is to test alpha 10 selected human MSC for their capacity to differentiate to a chondrocyte.

### A. Chondrogenic Differentiation

Clones isolated by alpha 10 expression according to example 4 are cultured in presence or absence of 10 ng/ml FGF2 (BioSource Europe SA, Belgium) for 14 days.

Clones are then induced to a chondrocyte phenotype in pellet mass culture with 2×10⁵ cells/pellet in DMEM (GibcoBRL, Paisley, UK) supplemented with 1× Insulin-transferrin sodium selenite (Sigma, Sweden), 0.1 μM dexamethasone (Sigma, Sweden), 50 μM ascorbic acid (Sigma, Sweden), 1 mg/ml Linoleic acid-bovine serum albumin (Sigma, Sweden), 1% Nonessential AA (GibcoBRL, Paisley, UK), 100 U/ml Penicillin, 100 μg/ml Streptomycin (GibcoBRL, Paisley, UK) and 10 ng/ml TGF-β3 (R&D Systems Europe Ltd., United Kingdom) as in example 3.

To determine the chondrogenic differentiation the pellet cultures are tested for Collagen type II, alpha 10-, and alpha 11-integrins, Sox 9 and aggrecane expression at mRNA level as described in Example 3. The synthesis of collagen II expression is also measured at protein level with a pro-collagen II ELISA (IBEX Technologies Inc., Montreal, Quebec, Canada) according to the manufactures description. The proteoglycan synthesis in pellet mass culture is measured by S³⁵ incorporation as in Example 3.

### Materials and Methods for Examples 6-10.

Isolation of Mesenchymal Stem Cells from Human Bone Marrow

Posterior iliac aspirations were performed on healthy volunteers for adult bone marrow (BM) collection, or on patients undergoing posterolateral spinal fusion surgery. The human bone marrow cells were diluted in equal amount of PBS (with Ca2+ and Mg2+) (GibcoBRL, Paisley, UK), 0.6% NaCitrate (Sigma, Sweden), 0.1% BSA (SERVA Electrophoresis GmbH, Heidelberg, Germany) and 100 U/ml DNase (Sigma, Sweden). The mononuclear cells (MNCs) were isolated by layering the bone marrow cells on a density gradient (Lymphoprep™, density 1.077 g/ml, Nycomed, Norway) accordingly to the manufactures descriptions. The MNC were washed twice in PBS and re-suspended in MEM α-medium (GibcoBRL, Paisley, UK) 20% FCS, 100 U/ml Penicillin and 100 μg/ml Streptomycin (GibcoBRL, Paisley, UK) and 1× Glutamax (GibcoBRL, Paisley, UTK) and cultured at a density of 0.8×10⁶ cells/cm². For the experiments in which integrin proteins were extracted and characterized, the MSCs were grown in DMEM with 10% FCS. For all cultures, after 4 days of culture non-adherent cells were removed by changing medium. Every 3-4 days the medium was changed and the cells were cultured until subconfluency and passaged at a density of about 5000 cells/cm². By day 7 after isolation, mesenchymal stem cells were cultured in presence or absence of 10 ng/ml FGF2 (BioSource Europe SA, Belgium).

### Aggregate Culture for Chondrogenic Differentiation

At confluency, MSCs were trypsinized and placed into aggregate cultures of 200,000 cells by centrifugation and cultured in 0.5 ml of a defined chondrogenic medium. The chondrogenic medium contained DMEM (GibcoBRL, Paisley, UK) supplemented with 1× Insulin-transferrin sodium selenite (Sigma, Sweden), 0.1 μM dexamethasone (Sigma, Sweden), 50 μM ascorbic acid (Sigma, Sweden), 1 mg/ml linoleic acid-bovine serum albumin (Sigma, Sweden), 1% nonessential AA (GibcoBRL, Paisley, UK), 100 U/ml penicillin, 100 μg/ml streptomycin (GibcoBRL, Paisley, UK) and 10 ng/ml TGFβ3 (R&D Systems Europe Ltd., United Kingdom).

### FFACS Analysis

The primary antibodies used were mAb365 mIgG₂ₐ (α10) (Cartela A B, Sweden), C09-biotin (α11) (BioInvent Int. AB, Sweden), CD44-PE (hyaluronan receptor) (BD, San Jose, Calif.), CD45-FITC mIgG1 (BD, San Jose, Calif.), CD49a-PE mIgG1 (α1) (BD, San Jose, Calif.), CD49b mIgG1 (α2) (BD, San Jose, Calif.), CD49e mIgG₁ (α5) (BD, San Jose, Calif.), CD51 mIgG₁ (αV) (Chemicon), CD29 mIgG₁ (β1) (P4C10), CD61 mIgG₁ (β3) (BD, San Jose, Calif.), CD90 mIgG₁ (Thy 1) (BD, San Jose, Calif.), CD105 mIgG₁ (endoglin) (BD, San Jose, Calif.), CD166 mIgG₁ (ALCAM) (BD, San Jose, Calif.) at a concentration of 1 μg/ml. The isotype control used were mIgG₂ₐ (Sigma, Sweden), mIgG₁ (BD, San Jose, Calif.) and CT17-biotin (Cartela A B, Sweden). The secondary antibodies used were Cy™5 conjugated anti-mIgG (Jackson ImmunoResearch, Pennsylvania) and PE conjugated Streptavidin (BD, San Jose, Calif.). The FACS stainings were done according to the manufacturer's instructions. The cell marker expression was detected with a FACSort (BD, San Jose, Calif.) and analyzed using the CellQuest® software (BD, San Jose, Calif.).

### Immunoblotting

Proteins were extracted from monolayer cells at time zero and from aggregates at day 1, 2, 3, 5, 7, and 10 by homogenization in a lysis buffer with the addition of protease inhibitors (Molecular Grinding Resin, Geno Technology). The amount of intracellular protein decreases proportionally during the differention of chondrocytes with the amount of synthesized extracellular matrix proteins. The amounts of cellular protein loaded were therefore adjusted to equal amounts by first immunoblotting for a cellular protein, GAPDH, and evaluating by quantitatively immunoblotting via ECF fluorimagery (Amersham-Molecular Dynamics). After determination of appropriate volumes, lysates were blotted for α1, α10, α11, and α1 integrin subunits and normalized to GAPDH levels using fluorimagery. Antibodies used were: polyclonal rabbit anti-human integrin α10 (Camper, L. et al., J Biol Chem, 1998. 273(32): p. 20383-9), rabbit anti-mouse integrin all (Zhang, W. M., et al., Matrix Biol, 2002. 21(6): p. 513-23), al, al, and GAPDH from Chemicon.

Stimulation with Different Growth Factors

MSCs were cultured for 4 weeks before adding growth factors for 5 days. 5×10⁴ MSCs cells/6-well plate were stimulated with 10 ng/ml FGF2 (BioSource Europe SA, Belgium), 10 ng/ml TGFβ₃ (R&D Systems Europe Ltd., United Kingdom), 100 ng/ml BMP2 (R&D Systems Europe Ltd., United Kingdom), 100 ng/ml BMP7 (R&D Systems Europe Ltd., United Kingdom) and 100 ng/ml IGF1 (R&D Systems Europe Ltd., United Kingdom). MSCs were cultured in MEM α-medium (GibcoBRL, Paisley, UK) 20% FCS, 100 U/ml Penicillin and 100 μg/ml Streptomycin (GibcoBRL, Paisley, UK) and 1× Glutamax (GibcoBRL, Paisley, UK). Growth factors were added at day 1 and 3 during the 5 days stimulation. Total RNA was isolated and used to study α10- and α11-integrin gene expression with Quantitative-PCR.

For the kinetic studies 5×10⁴ MSCs cells/well were stimulated in 6 well plates with 10 ng/ml FGF2 during 6, 4, 2, 1 and 0 days. At the end of the experiment the results were analysed with FACS and Quantitative-PCR.

### Immunostaining

Hindlimbs from 8-week old α10 integrin knockout mice and their control littermates were decalcified (in 10% EDTA, 7,5% polyvinylpyrrolidine, in 0.1M Tris, 2 tablespoons KOH at pH 6,95) for 1 week, frozen in OCT and cut at 6 um sections. Immunostaining was performed as described in Bengtsson et al., (2005, J Cell Sci, 2005. 118(T 5): p. 929-36). Antibodies used were: polyclonal rabbit anti-human integrin α10 (Camper et al., 1998, J Biol Chem, 1998. 273(32): p. 20383-9) and rabbit anti-mouse integrin all (Popova et al., 2003). Secondary antibody used was: biotinylated goat anti rabbit IgG (from Vector Laboratories Inc., CA). Vectastain ABC and Vector VIP reagent were from Vector Laboratories Inc., CA and Methyl Green for counterstaining was from Sigma.

### Collagen II and Proteoglycan Synthesis

Collagen type II synthesis was measured at protein level with a procollagen II ELISA (IBEX Technologies Inc., Montreal, Quebec, Canada) according to the manufactures description. Proteoglycan synthesis was measured by metabolic labelling with ³⁵S. Pellets where pulsed with 50 μCi/ml ³⁵S for 4 hours, washed with 200 μl PBS and digested over night with 10 U Papain (SIGMA #P3125) in 200 μl 100 mM NaAc, 10 mM Cysteine-hydrochloride, 2 mM EDTA, pH˜5.5. Free isotope was removed by precipitation of proteoglycan with hexadecylpyridinium chloride monohydrate (SIGMA# C5460) at a final concentration of 30 mM in the presence of 100 μg/ml chondroitin sulphate-6 (SIGMA# C4384). The precipitate was collected by centrifugation at 5000×g for 10 minutes and subsequently washed two times with precipitation buffer before it was finally dissolved in concentrated formic acid and taken to counting in a β-counter.

### Quantitative PCR

Total RNA was isolated with Qiagen RNeasy (QIAGEN, GmbH, Germany) according to the manufactures protocol. Total RNA (2 μg) was reverse transcribed by Superscript II (200 units) (Invitrogen™ Life Technologies, Carlsbad, Calif.) using random hexamer oligonucleotides. The quantitative PCR was performed using the LightCycler® FastStart DNA Master SYBR GreenI (Roche Applied Science, Mannheim, Germany). All PCRs were performed at a thermal profile of 95° C. for 10 min, 95° C. for 10 s, 65° C. 5 s, 72° C. 15 s. Primers are given in table 2.

### Migration

The FGF2 treated and untreated MSCs were studied for their migratory capacity in well chambers (Neuroprobe Inc. Gaitersburg, Md., USA). The PFA(Polyvinylpyrrolidone-free polycarbonate)-membranes (pore size 0.8 μM were coated with 10 μg/ml collagen type II in PBS for 30 minutes on both sides before 5×10⁴ cells were added to each membrane in MEM α-medium with 1% BSA. The cells (in three parallel wells) were allowed to migrate towards 0 and 2% serum over night. The filters were fixed with 10% MeOH, stained with haematoxylin and mounted on glass slides. Non-migrating cells were wiped away before photos of three independent areas of each membrane were taken using a Nicon Eclipse TE2000-S microscope and a Nicon digital camera. The relative amount migrating cells were analysed using the Visiopharm software (Visiopharm A/S, Copenhagen, Denmark).

### Example 6

### Expression of Collagen Binding Integrins on Monolayer Cultured MSC

### Objective

The objective of this example is to show that MSCs in monolayer cultures are highly positive for collagen binding integrins.

### Material and Methods

Material and methods are given in the method section above.

### Results

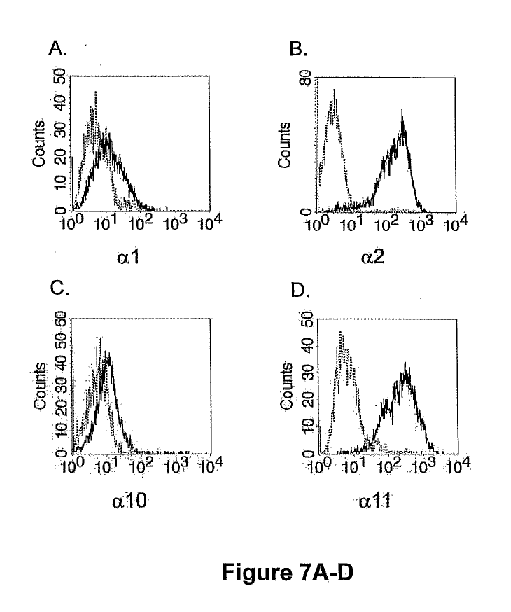

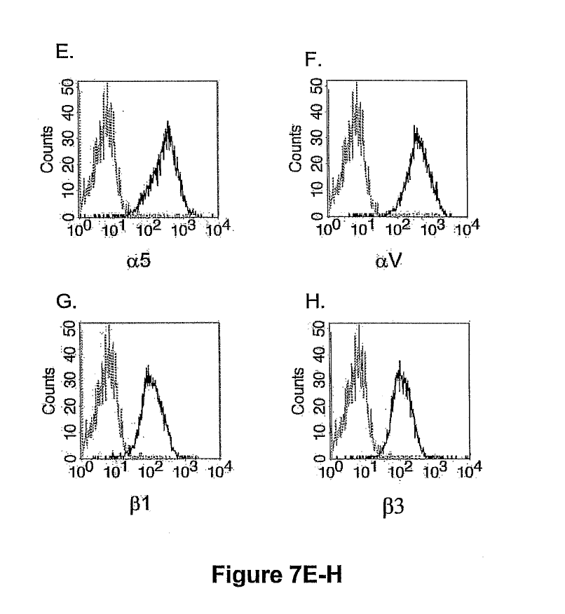

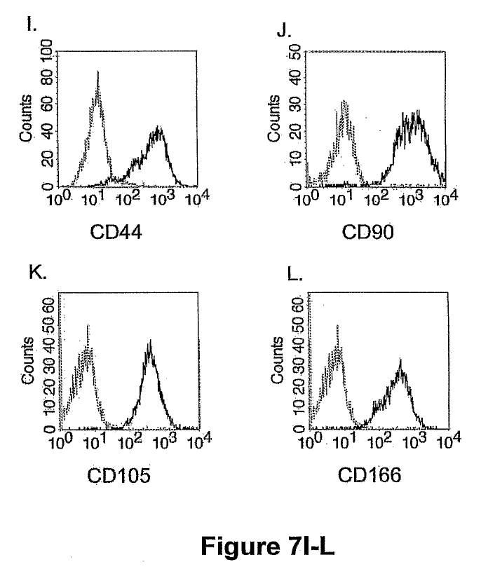

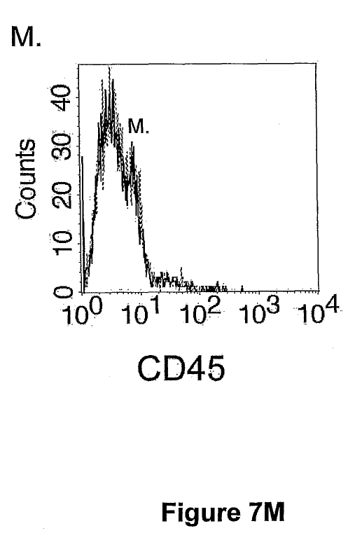

Human bone marrow MSCs, isolated by plastic adherence, were characterised by FACS, for the expression of different cell surface markers, focusing on integrin subunit expression after 21 days in monolayer culture. Among the MSCs, different cell populations cells could be found that are positive for the four known collagen binding integrins α1-, α2-, α10- and α11β1. The cells expressed α1 (35%), α2 (95%), α10 (38%) and α11 (930%) (FIG. 7A-D). The MSCs were also positive for αV (99%, FIG. 7F), normally expressed on skeletal muscle, and the fibronectin receptor integrin α5 (98%, FIG. 7E). Further, the cells stained positive for the integrin subunit β1 (96%, FIG. 7G) and β3 (98%, FIG. 7H). The MSC preparations were also characterized as negative for the leukocyte marker CD45 (FIG. 7M) and positive for several markers commonly used in characterizing MSCs, i.e. CD105 (99%, FIG. 7K), CD166 (98%, FIG. 7L), CD44 (91%, FIG. 7I) and CD90 (97%, FIG. 7J). However, MSCs cultured in monolayer have no detectable expression of collagen type II mRNA (data not shown).

### Example 7

### FGF2 and TGFβ₃ Stimulation Polarize α10 and α11 Integrin Expression

### Objective

The objective of this example is to show that FGF2 and TGFβ₃ stimulation polarize α10 and α11 integrin expression.

### Results

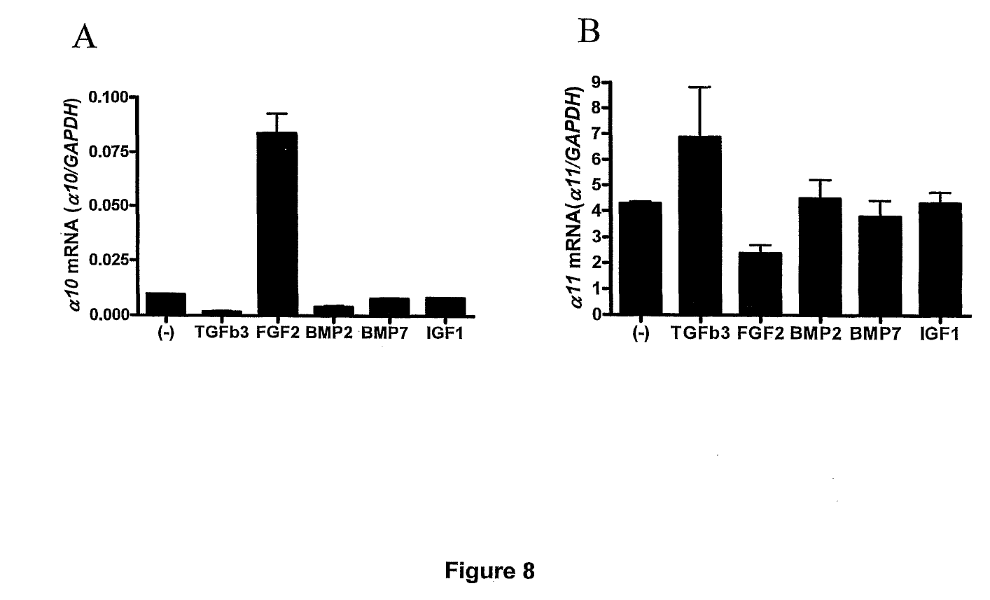

To investigated whether different growth factors could regulate the integrin expression of MSCs, cells were cultured in monolayer in the presence of TGFβ3, FGF2, BMP2, BMP7 or IGF1 for five days before the mRNA expression of integrin subunits α10 and α11 were analysed using Q-PCR (FIG. 8). MSCs cultured in monolayer for five days in the presence of FGF2 had a 8-fold increase in mRNA expression of integrin subunit α10 compared to untreated cells (FIG. 8A). In contrast, the FGF2 treatment resulted in a decreased expression of integrin subunit all compared to untreated cells (FIG. 8B). TGFβ3 treatment of MSC for five days had the opposite effect; lowering the integrin α10 (FIG. 8A) and increasing all (FIG. 8B) mRNA expression. BMP2, BMP7 and IGF1 treatment had no effect on the mRNA expression of integrin subunit α10 or α11.

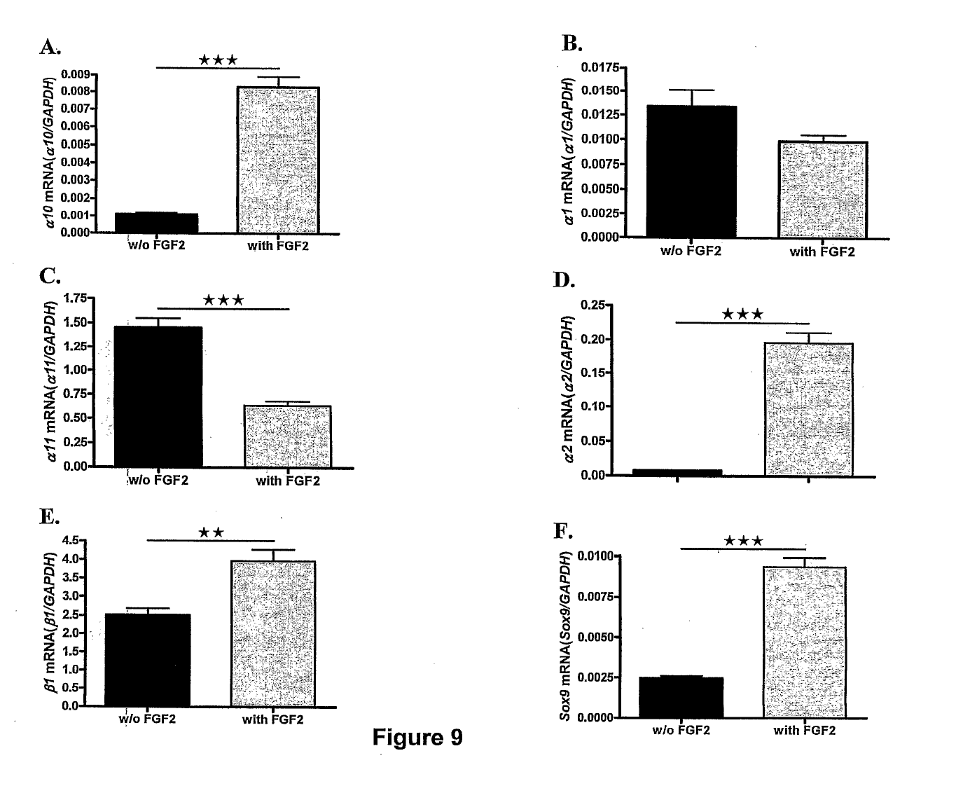

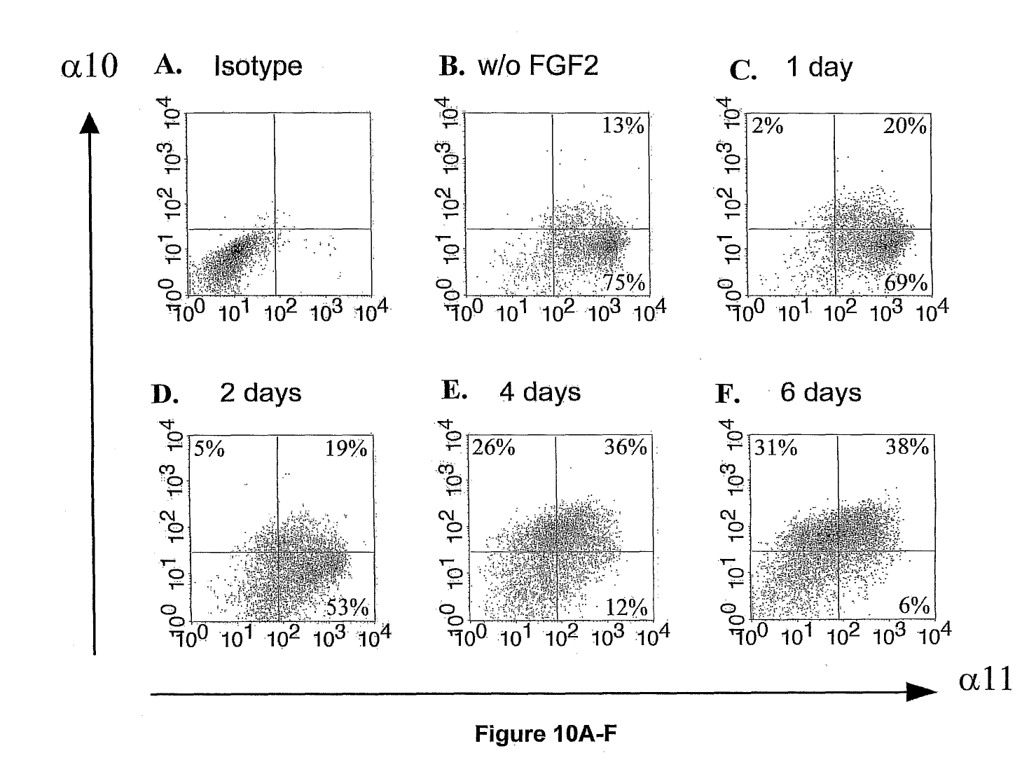

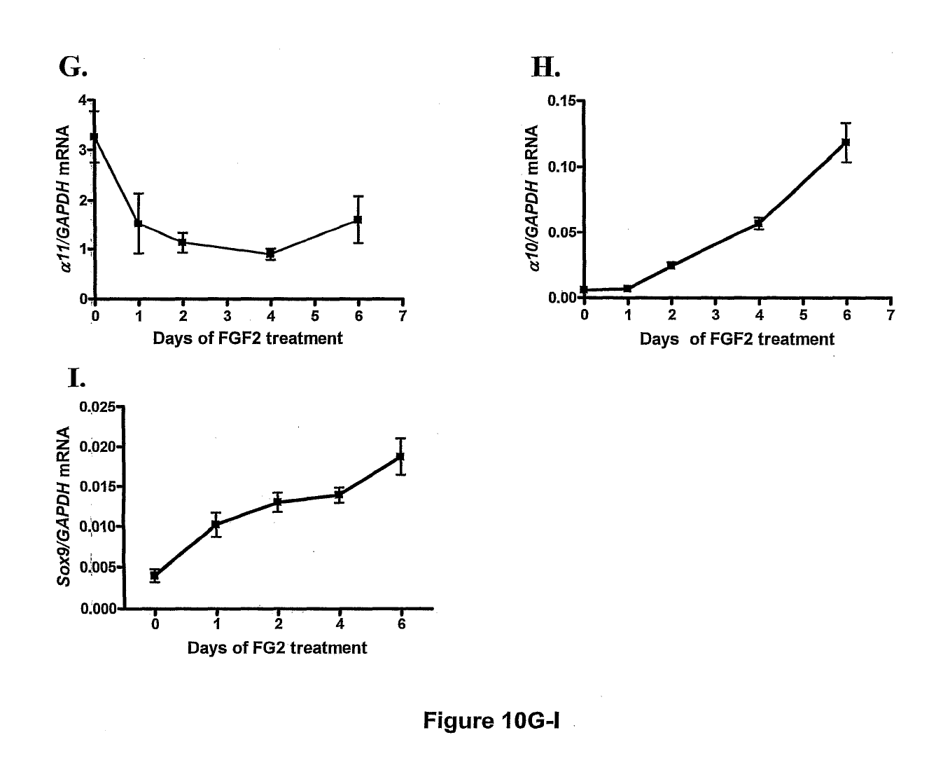

To further assess the effect of FGF2, MSCs prepared from a different donor were stimulated with FGF2 for five days in monolayer culture and the mRNA expression of α1, α2, α10, α11, β1, Sox9 and COL2A1 was quantified using real-time PCR (FIG. 9). The expression of α2, α10 and Sox9 increased (Mann-Whitney test p<0.001, n=8) as did that of β1 (Mann-Whitney test p<0.01, n=8). In contrast, all expression decreased (Mann-Whitney test p<0.001, n=8) with FGF2 treatment. The expression of α1 was not significantly changed and COL2 μl was not detected (data not shown). MSCs were then treated for 1, 2, 4, and 6 days with FGF2 to evaluate the kinetics of the FGF2 effect on α10 expression. FGF2 treatment increased the percentage of α10 positive MSCs from 13% to 69% during the 6 days of culture (FIG. 10A-F), while the percentage of α11 positive MSCs decreased from 88% to 44%. The double positive (α10 and α11) cell population increased during the same conditions from 13% to 38%. At the RNA level, α10 and Sox9 increased while all decreased with FGF2 treatment, supporting the FACS results (FIG. 10G-I).

### Example 9

### Culture of MSCs with FGF2 Induces Stable Expression of α10

### Objective

The objective of this example is to show that culture of MSCs with FGF2 induces stable expression of α10.

### Results

Our results showed that treatment with FGF2 can counter-regulate α10 and α11 expression in human MSCs cultured in monolayer. These findings prompted us to evaluate the potential effect of prolonged FGF2 treatment in these cells.

MSCs cultured with or without FGF2 in monolayer were analysed for the expression of α10, α11 and β1 integrins as well as for CD105 (endoglin) and CD166 (ALCAM) at day 14, 28 and 50 (FIG. 11A-D). The expression of α10 and α11 was not detectable by FACS analysis in total bone marrow (BM) cells directly after preparation i.e. day 0. Approximately 10% of these cells are positive for α1, α2, β1 and CD105, while 20% of freshly isolated BM cells are positive for CD166 (data not shown). Already at day 14 close to 100% of the cells showed a stable expression of CD105 and CD166 and the expression was unaffected by the FGF2 treatment and stable during the culture time. The expression of α10 and α11 are earliest detected at the first passage, around day 10. At day 14 approximately 70% of the FGF2 treated MSCs were α10 positive, in comparison only 10-20% of the untreated cells stained positive for α10 (FIG. 6A) after 14 days in culture. The FGF2 treated cultures generated a larger population of α10 expressing cells (70-80%) during the entire culture period of 50 days. We conclude that integrin α10 is a cell-surface marker of FGF2 treated MSC cultures.

### Example 10

### Immunohistochemistry of Alpha10 Expression in Endosteum and Periosteum

### Objective

The objective of this example was to analyse the frequency of alpha10 and alpha11 expressing BM cells.

### Results

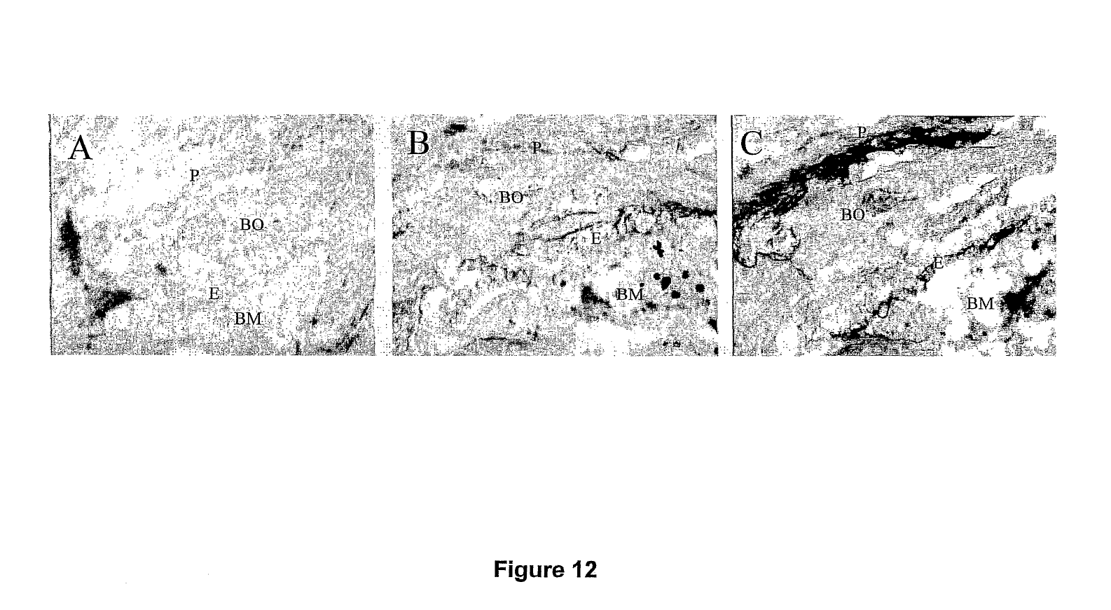

Immunohistochemistry revealed α10 expression in the endosteum (the cell lining between the bone marrow and bone) and a lower expression in the periosteum (the cell lining outside the bone) (FIG. 12). The opposite staining pattern was seen with the α11 specific antibodies, i.e. a weak expression in the endosteum and stronger in the periosteum. Both endosteum and periosteum are tissues where mesenchymal progenitor cells can be found. The immunohistochemical analysis did not detect expression of α10 or α11 positive cells in the bone marrow; however we cannot exclude the possibility that, due to low cell frequency, we were unable to detect them.

### Example 11

### High α10 Expression is Correlated with Better Chondrogenic Differentiation Potential

### Objective

The objective of this example is to show that high α10 expression is correlated with better chondrogenic differentiation potential.

### Results

The present study shows that the α10 expression is higher in FGF2 treated cell populations. This is of special interest since the α10 integrin has been described as a chondrocyte-specific marker. Therefore, it was hypothesized that cells with higher α10 expression would have a greater differentiation potential towards a chondrogenic phenotype. To address this hypothesis, human MSCs cultured with or without FGF2, were subjected to chondrocyte differentiation in aggregate cultures. The FGF2 treated and untreated cells were verified to be integrin α10-high/α11-low and α10-low/α11-high respectively by FACS analyses, before exposing them to differentiation conditions (data not shown). Total-RNA was extracted from pellets on day 7, day 14 and day 21 after onset of pellet-mass culture and quantitatively analyzed for gene-expression of GAPDH, collagen type II, aggrecan, versican, integrin α10, integrin α11 and Sox9. Supernatants were collected and analyzed for newly synthesized collagen type II (CPII pro-peptide). Separate pellets were analyzed for proteoglycan synthesis using 35-S incorporation.

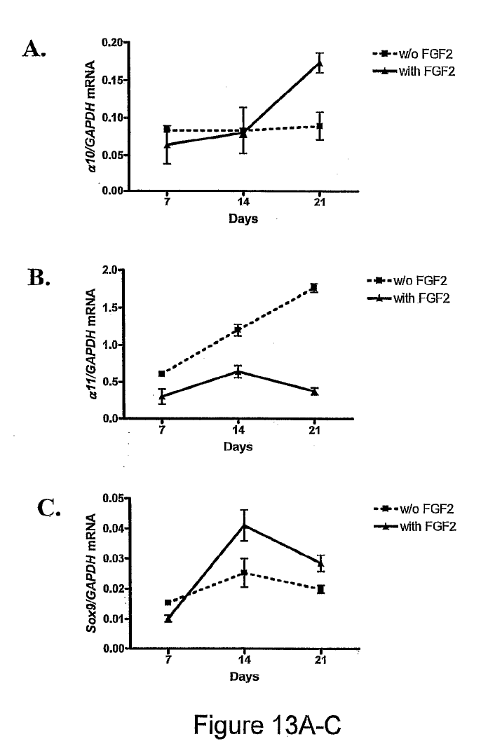

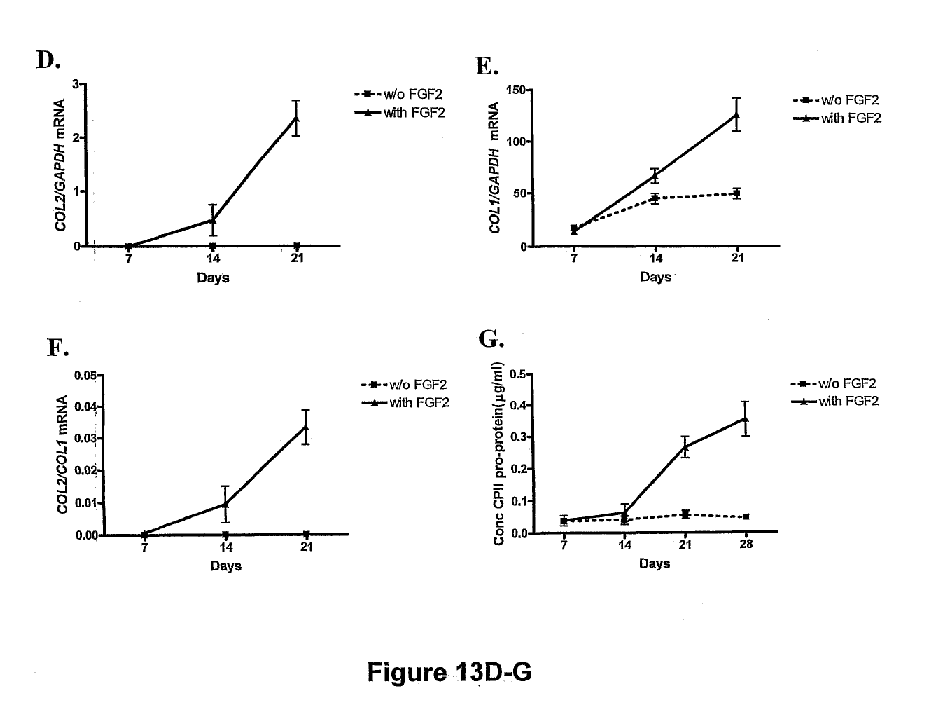

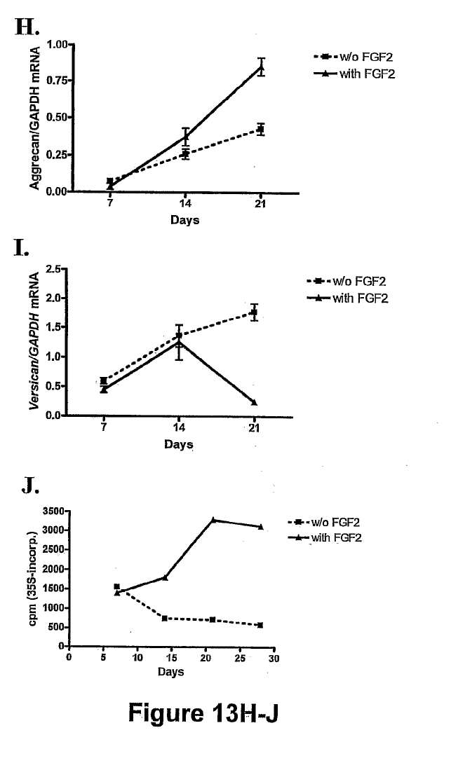

The results showed that aggregate cultures of FGF2 treated cells had an increased mRNA expression of collagen type II (COL2A) already at day 14, which further increased at day 21. The aggregate cultures of FGF2 untreated cells expressed very low levels of collagen type II (FIG. 13A). By analyzing the supernatants from the aggregate cultures for newly synthesized collagen type II protein (i.e. CPII pro-peptide), we could verify that the FGF2 treated MSCs synthesize and process collagen type II (FIG. 13G). The aggregate cultures from untreated MSCs did not synthesize detectable levels of CPII pro-peptide.

FGF2 treated cells had an increased mRNA expression of aggrecan and a decreased expression of versican compared to the un-treated cells (FIGS. 13H and 13I). The expression of aggrecan increases over time reaching maximum levels at day 21. We also analyzed proteoglycan synthesis (i.e. 35-S incorporation) at different time-points during pellet formation (FIG. 13J). The results show that FGF2 treated MSCs had increased proteoglycan mRNA and protein levels compared to the untreated cells, and that the proteoglycan synthesis peaks around day 21 (FIG. 13I).

The expression levels of integrin α10 and Sox9 mRNA were also increased in the aggregate cultures of FGF2 treated cells compared to the untreated cells, while α11 mRNA levels were higher in FGF2 untreated MSCs aggregate cultures (FIG. 13 A-C).

We conclude from the gene expression profile that FGF2 treatment of MSCs increases their chondrogenic potential, which is also signified by the high α10 integrin expression of this cell population.

### Experiment 12

### FGF2 Treatment Increases the Migratory Potential of MSCs

### Objective

The objective with this example is to evaluate the effect of FGF2 treatment on the migratory potential of MSCs.

### Results

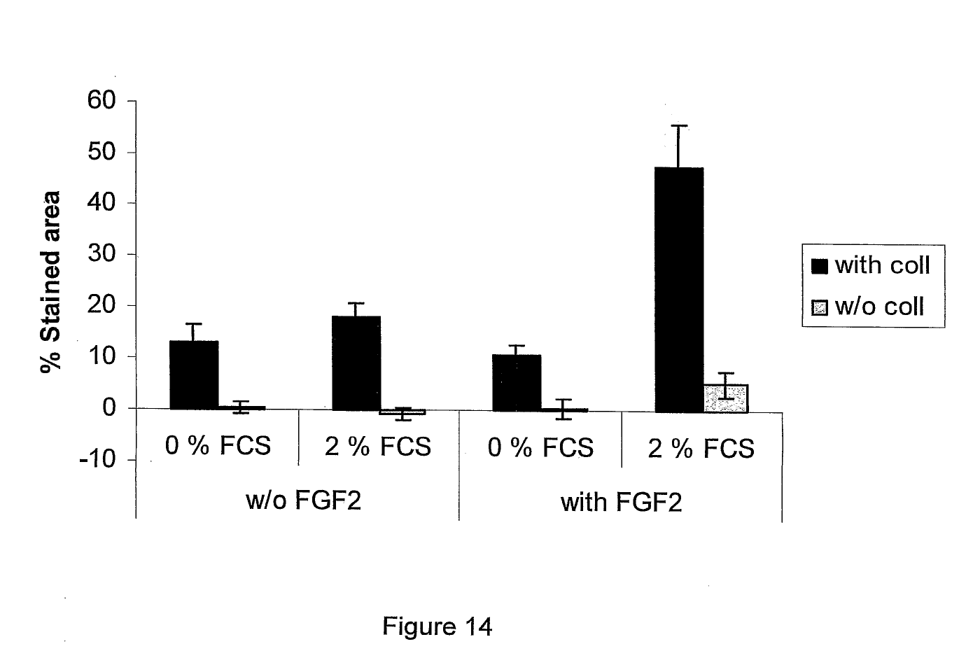

Since FGF2 treatment changes the expression profile of collagen binding integrins on MSCs, we hypothesized that FGF2 treatment would change the migratory potential of MSCs on collagen. To address this question we analysed FGF2 treated and untreated MSC-populations in a modified Boyden chamber analysis using collagen type II coated membranes. 78% of the FGF2 treated cells and 18% of the untreated cells stained positive for α10 at the onset of the migration experiments (Data not shown). We could demonstrate that both FGF2 untreated and treated MSCs could migrate towards a serum gradient on collagen type II and that the migration was collagen dependant. Interestingly, the FGF2 treated cells, with a higher integrin α10 expression, had an increased migratory potential on collagen type II compared to the untreated cells (FIG. 14).

1. A cell culture system for differentiating mammalian MSC to chondrocytes, the cell culture system comprising
a) a population of isolated mammalian MSC,
b) optionally, at least one additive promoting expansion of said mammalian MSC, wherein the at least one additive promoting expansion of said mammalian MSC is a FGF family protein, and
c) at least one additive promoting differentiation to chondrocytes,
wherein the MSC are selected for expression of integrin alpha10.

2. The cell culture system of claim 1, wherein the at least one additive promoting differentiation to chondrocytes is a member of the TGF beta super family proteins.

3. The cell culture system of claim 2, wherein the member of the TGF beta super family is TGF beta 3.

4. The cell culture system of claim 3, wherein FGF is FGF2.

5. The cell culture system according to claim 1, wherein FGF2 and TGFβ3 are added sequentially to the system and the MSC thereby cultured sequentially in the presence of said additive, and wherein adding of FGF2 is preceding TGFβ3 addition.

6. The cell culture system according to claim 1, wherein the MSC are selected for expression of integrin alpha10 before addition and culture of the MSC in the presence of FGF2.

7. The cell culture system according to claim 1, wherein the MSC are selected for expression of integrin alpha10 after addition and culture of the MSC in the presence of FGF2, but before addition of TGFβ3.

8. The cell culture system according to claim 1, wherein the MSC are selected for expression of integrin alpha10 after addition and culture the MSC in the presence of TGFβ3.

9. The cell culture system according to claim 1, wherein the chondrocyte has a phenotype comprising expression of alpha10, sox9, aggrecan, and collagen II.

10. The cell culture system according to claim 1, wherein FGF2 is added in an amount of about 0.01-1 μg/ml.

11. The cell culture according to claim 1, wherein TGFβ3 is added in an amount of about 0.01-1 μg/ml.

12. The cell culture system according to claim 1, wherein the mammalian MSC are human MSC.

13. A method of producing a substantially homogenous population of mammalian chondrocytes, expanded and differentiated from an isolated mammalian subset of MSC, the method comprising the steps of
a) providing a population of isolated MSC
b) optionally culturing the isolated MSC in a) above in the presence of at least one additive promoting expansion, wherein the at least one additive promoting expansion of said MSC is a FGF family protein, and
c) culturing the isolated MSC in the presence of at least one additive promoting differentiation to a chondrocyte,
wherein the MSC are selected for expression of integrin alpha10 expression.

14. The method according to claim 13, wherein the MSC are selected for expression of alpha10 expression before culturing in the presence of at least one additive promoting expansion.

15. The method according to claim 13, wherein the MSC are selected for expression of alpha10 expression after culturing in the presence of at least one additive promoting expansion, but before culturing in the presence of an additive promoting differentiation.

16. The method according to claim 13, wherein the MSC are selected for expression of alpha10 expression after culturing in the presence of at least one additive promoting differentiation.

17. The method according to claim 13, wherein the additive promoting differentiation is TGF beta 3.

18. The method according to claim 13, wherein said culture in the presence of FGF2 is for about 1 to 6 weeks.

19. The method according to claim 9, wherein said culture in the presence of TGFβ3 is for about 1-7 days.

20. The method according to claim 9, wherein FGF2 is added in an amount of about 0.01 ng/ml-1 μg/ml.

21. The method according to claim 9, wherein TGFβ3 is added in an amount of about 0.01 ng/ml-1 μg/ml.

22. The method according to claim 13, wherein the mammalian MSC are human MSC.

23. The method according to claim 13, wherein the substantially homogenous population include at least 60%, 70%, 80%, 90%, 95, 97, 99, 99.5 or even 99.9% expanded and differentiated MSC with a chondrocyte phenotype.

24. The method according to claim 9, wherein the chondrocyte has a phenotype comprising expression of alpha10, sox9, aggrecan, and collagen II.

25. The method according to claim 13, wherein the MSC are selected with beads.

26. The method according to claim 13, wherein the MSC are selected by fluorescent cell sorting.

27. The method according to claim 13, wherein the method comprises the cell culture system.

28. An isolated and substantially homogenous cell population of expanded and differentiated mammalian MSC, wherein said MSC has a chondrocyte phenotype, and wherein the cell population is obtained by the method of claim 13.

29. The cell population according to claim 28, wherein the chondrocyte phenotype comprises expression of alpha10, sox9, aggrecan, and collagen II.

30. The cell population according to claim 28, wherein the isolated and substantially homogenous population comprises at least 60, 70, 80, 90, 95, 97, 99, 99.5, or even 99.9% expanded and differentiated MSC cells with a chondrocyte phenotype.

31-33. (canceled)

34. A method for reconstituting cartilage, the method comprising administering an expanded and differentiated cell population according to claim 28 to a patient in the need thereof, wherein the cell population is transplanted/administered in an amount effective reconstitute cartilage tissue.

35. A method of treating a cartilage condition, the method comprising transplanting an isolated and substantially homogenous cell population of expanded and differentiated mammalian MSC for reconstitution of cartilage, wherein said MSC has chondrocyte phenotype, the method comprising administering an expanded and differentiated cell population according to claim 28 wherein the cell population is administered in an amount effective to reconstitute cartilage tissue and to treat said cartilage condition.

36. A pharmaceutical composition comprising an expanded and differentiated cell population according to claim 28 and a pharmaceutical acceptable carrier.

37. A kit for expanding and differentiating isolated mammalian MSC to a chondrocyte phenotype comprising the culture system according to claim 1.

38. (canceled)

39. A kit comprising an expanded and differentiated cell population according to claim 28.

40. The kit according to claim 39 further comprising means for delivering the cell population to a patient in the need thereof.

41. The kit according to claim 39, further comprising means for determining that the delivered cell population locate to at least one desired site.

42. The kit according to claim 41, wherein the desired site is cartilage.

43. A kit for reconstitution of cartilage, the kit comprising
a) an expanded and differentiated cell population according to claim 28,
b) means for reconstituting cartilage, and
c) optionally instructions for reconstituting cartilage.

44. (canceled)

45. A method of identifying a subpopulation of MSC with enhanced chondrogenic potential, the method comprising,
a) isolating a population of cells comprising MSC,
b) detecting integrin alpha10 expression on a subpopulation of said MSC,
c) comparing the alpha10 expression to a control cell population not expressing alpha10,
d) identifying alpha10 expressing MSC cells as subpopulation of MSC with an enhanced chondrogenic potential.

46. The method of claim 45, wherein the detection of alpha10 is done by immunological means.

47. The method according to claim 46, wherein the immunological means comprises adding an antibody specifically reacting with alpha10.

48. The method of claim 45, wherein the detection of alpha10 is done by identifying expression of the alpha10 gene.

49. The method of claim 48, wherein the detection of alpha10 gene expression is done by identifying RNA-transcripts of the alpha10 gene.
